(19) United States
(12) Patent Application Publication
(10) Pub. No.: US 2006/0052597 A1
(43) Pub. Date: Mar. 9, 2006

(54) Aryloxyalkylamine derivatives as h3 receptor ligands

(76) Inventors: Desmond John Best, Essex (GB); Gordon Bruton, Essex (GB); Thomas Daniel Heightman, Essex (GB); Barry Sidney Orlek, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/532,371

(22) PCT Filed: Oct. 20, 2003
(86) PCT No.: PCT/EP03/11649

§ 371 Date: Apr. 21, 2005

(30) Foreign Application Priority Data
Oct. 22, 2002 (GB) 0224558.7
Oct. 23, 2002 (GB) 0224677.5
Oct. 23, 2002 (GB) 0224678.3
Oct. 23, 2002 (GB) 0224679.1
Oct. 24, 2002 (GB) 0224783.1
Feb. 14, 2003 (GB) 0303467.5

(51) Int. Cl. C07D 413/02; C07D 403/02

(52) U.S. Cl. 544/122; 544/360; 544/230; 544/372

(57) ABSTRACT

The present invention relates to novel benzyloxy derivatives having pharmacological activity, processes for their preparation, to compositions containing them and to their use in the treatment of neurological and psychiatric disorders.

The present invention relates to novel phenoxy derivatives having pharmacological activity, processes for their preparation, to compositions containing them and to their use in the treatment of neurological and psychiatric disorders.

WO 02/76925 (Eli Lilly), WO 00/06254 (Societe Civile Bioprojet), WO 01/66534 (Abbott Laboratories) and (WO 03/004480 (Novo Nordisk) describe a series of compounds which are claimed to be histamine H3 antagonists. WO 02/40466 (Ortho McNeill Pharmaceutical) disclose a series of amido-alkyl piperidine and amido-alkyl piperazine derivatives which are claimed to be useful in treatment of various nervous system disorders.

The histamine H3 receptor is predominantly expressed in the mammalian central nervous system (CNS), with minimal expression in peripheral tissues except on some sympathetic nerves (Leurs et al., (1998), Trends Pharmacol. Sci. 19, 177-183). Activation of H3 receptors by selective agonists or histamine results in the inhibition of neurotransmitter release from a variety of different nerve populations, including histaminergic and cholinergic neurons (Schlicker et al., (1994), Fundam. Clin. Pharmacol. 8, 128-137). Additionally, in vitro and in vivo studies have shown that H3 antagonists can facilitate neurotransmitter release in brain areas such as the cerebral cortex and hippocampus, relevant to cognition (Onodera et al., (1998), In: The Histamine H3 receptor, ed Leurs and Timmerman, pp255-267, Elsevier Science B.V.). Moreover, a number of reports in the literature have demonstrated the cognitive enhancing properties of H3 antagonists (e.g. thioperamide, clobenpropit, ciproxifan and GT-2331) in rodent models including the five choice task, object recognition, elevated plus maze, acquisition of novel task and passive avoidance (Giovanni et al., (1999), Behav. Brain Res. 104, 147-155). These data suggest that novel H3 antagonists and/or inverse agonists such as the current series could be useful for the treatment of cognitive impairments in neurological diseases such as Alzheimer's disease and related neurodegenerative disorders.

The present invention provides, in a first aspect, a compound of formula (I) or a pharmaceutically acceptable salt thereof: 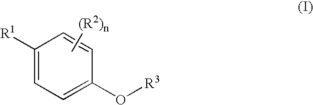
wherein:
R¹ represents a group of formula (A): 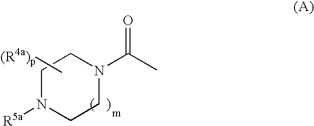
   wherein R^{4a} represents C₁₋₆alkyl, oxo, aryl, heteroaryl or heterocyclyl; R^{5a} represents hydrogen, -C₁₋₆ alkyl, -C₁₋₆alkylC₁₋₆alkoxy, -C₁₋₆alkoxycarbonyl, -C₃₋₈ cycloalkyl, -aryl, -heterocyclyl, heteroaryl, -C₁₋₆ alkyl-aryl, —CH(aryl)(aryl), -C₁₋₆ alkyl-Cm cycloalkyl, -C₁₋₆ alkyl-heteroaryl or -C₁₋₆ alkyl-heterocyclyl, wherein R^{5a} may be optionally substituted by one or more (eg. 1, 2 or 3) substituents which may be the same or different, and which are selected from the group consisting of halogen, hydroxy, cyano, nitro, oxo, haloC₁₋₆ alkyl, polyhaloC₁₋₆alkyl, haloC₁₋₆ alkoxy, polyhaloC₁₋₆alkoxy, C₁ alkyl, C₁₋₆ alkoxy, C₁₋₆alkylthio, C₁₋₆ alkoxyC₁₋₆alkyl, C₃₋₇ cycloalkylC₁₋₆ alkoxy, C₁₋₆alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, C₁₋₆ alkylsulfonamidoC₁₋₆ alkyl, C₁₋₆ alkylamidoC₁₋₆alkyl or a group NR^{15a}R^{16a}, —CONR^{15a}R¹⁶, —NR⁵²COR^{16a}, NR^{15a}SO₂R^{16a} or —SO₂NR^{15a}R^{16a}, wherein R^{15a} and R^{16a} independently represent hydrogen, C₁₋₆ alkyl, aryl or together with the nitrogen to which they are attached may form a nitrogen containing heterocyclyl group;
m is 1 or 2;
p is 0, 1, 2 or 3, or when p represents 2, said R^{4a} groups may instead form a bridging group consisting of one or two methylene groups;
or R¹ represents a group of formula (B): 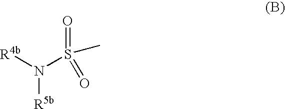
   wherein NR^{4b}R^{5b} represents an N-linked -heterocyclyl, -heterocyclyl-X^{b}-aryl, -heterocyclyl-X^{b}-heteroaryl, -heterocyclyl-X^{b}-heterocyclyl, -heteroaryl, -heteroaryl-X^{b}-aryl, -heteroaryl-X^{b}-heteroaryl or -heteroaryl-X^{b}-heterocyclyl group; wherein said aryl, heteroaryl and heterocyclyl groups of NR^{4b}R^{5b} may be optionally substituted by one or more (eg. 1, 2 or 3) substituents which may be the same or different, and which are selected from the group consisting of halogen, hydroxy, cyano, nitro, oxo, haloC₁₋₆ alkyl, polyhaloC₁₋₆ alkyl, haloC₁₋₆ alkoxy, polyhaloC₁₋₆ alkoxy, C₁₋₆ alkyl, C₁₋₆alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆alkoxycarbonyl, arylC₁₋₆ alkyl, heteroarylC₁₋₆ alkyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆alkyl, aryloxy, C₁₋₆alkylsulfonamidoC₁₋₆alkyl, C₁₋₆ alkylamidoC₁₋₆alkyl, arylsulfonamido, arylaminosulfonyl, arylsulfonamidoC₁₋₆ alkyl, arylcarboxamidoC₁₋₆alkyl, aroylC₁₋₆alkyl, arylC₁₋₆ alkanoyl, or a group —NR^{15b}R^{16b}, —CONR^{15b}R^{16b}, NR^{15b}COR^{16b}, —NR^{15b}SO₂R^{16b} or —SO₂NR^{15b}R^{16b}, wherein R^{15b} and R^{16b} independently represent hydrogen or C₁₋₆ alkyl;
X^{b} represents a bond, CO, NHCO or CONH;
or R¹ represents a group of formula (C): 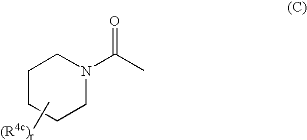
   wherein R^{4c} represents C₁₋₆ alkyl, OH, aryl or heterocyclyl, wherein said aryl and heterocyclyl groups may be optionally substituted by halogen, C₁₋₆alkyl, C₁₋₆alkoxy, cyano, amino, oxo, trifluoromethyl or an aryl group;
r is 0, 1 or 2;
or R¹ represents a group of formula (D): 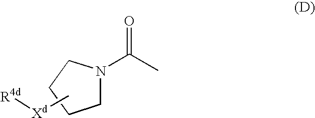
   wherein R^{4d} represents aryl or heteroaryl wherein said aryl and heteroaryl groups may be optionally substituted by one or more (eg. 1, 2 or 3) substituents which may be the same or different, and which are selected from the group consisting of halogen, C₁₋₆alkyl, C₁₋₆alkoxy, cyano, amino or trifluoromethyl;
X^{d} represents a bond or NHCO, such that when X^{d} represents NHCO, the group R^{4d}-X^{d} is attached at the 3-position of the pyrrolidinyl ring;
   or R¹ represents a group of formula —CO-E, wherein E represents a group of formula E^{a}, E^{b} or E^{c}: 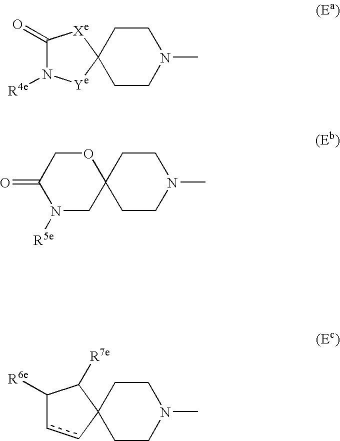
      wherein X^{e} represents O or N—R^{8e};
Y^{e} represents —C(HR^{9e})— or —C(═O)—;
R^{4e}, R^{5e}, R^{8e} and R^{9e} independently represent hydrogen, C₁₋₆ alkyl, aryl, heteroaryl, -C₁₋₆alkyl-aryl or -C₁₋₆ alkyl-heteroaryl;
R^{6e} and R^{7e} independently represent hydrogen, C₁₋₆ alkyl, aryl, heteroaryl, -C₁₋₆ alkyl-aryl, -C₁₋₆ alkyl-heteroaryl or R^{6e} and R^{7e} together with the carbon atoms to which they are attached may form a benzene ring;
[symbol] is a single or double bond;
wherein said aryl or heteroaryl groups of R^{4e}, R^{5e}, R^{6e}, R^{7e}, R^{8e} and R^{9e} may be optionally substituted by one or more (eg. 1, 2 or 3) substituents which may be the same or different, and which are selected from the group consisting of C₁₋₆ alkyl, CF₃, C₁₋₆ alkoxy, halogen, cyano, sulfonamide or C₁₋₆ alkylsulfonyl;
or R¹ represents a group of formula (F): 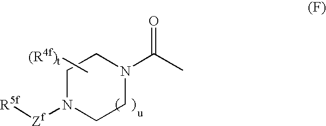
wherein t is 0, 1 or 2;
u is 1 or 2;
R^{4f} represents C₁₋₆ alkyl or when t represents 2, said R^{4f} groups may instead form a bridging group consisting of one or two methylene groups;
R^{5f} represents -C₁₋₆ alkyl, -C₁₋₆ alkylC₁₋₆ alkoxy, -C₃₋₄ cycloalkyl, aryl, heterocyclyl, heteroaryl, -C₁₋₆ alkyl-aryl, -C₁₋₆ alkyl-C₁₋₆cycloalkyl, -C₁₋₆ alkyl-heteroaryl, -C₁₋₆ alkyl-heterocyclyl, -aryl-aryl, -aryl-heteroaryl, -aryl-heterocyclyl, -heteroaryl-aryl, -heteroaryl-heteroaryl, -heteroaryl-heterocyclyl, -heterocyclyl-aryl, -heterocyclyl-heteroaryl or -heterocyclyl-heterocyclyl;
wherein R^{5f} may be optionally substituted by one or more (eg. 1, 2 or 3) substituents which may be the same or different, and which are selected from the group consisting of halogen, hydroxy, cyano, nitro, oxo, haloC₁₋₆ alkyl, polyhaloC₁₋₆ alkyl, haloC₁₋₆ alkoxy, polyhaloC₁₋₆ alkoxy, C₁₋₆ alkyl, C₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, C₁₋₆ alkylsulfonamidoC₁₋₆ alkyl, C₁₋₆ alkylamidoC₁₋₆ alkyl, arylsulfonyl, arylsulfonyloxy, aryloxy, arylsulfonamido, arylcarboxamido, aroyl, or a group NR^{15f}R^{16f}, —CONR^{15f}R^{16f}, —NR^{15f}COR^{16f}, —NR^{15f}SO₂R^{16f} or —SO₂NR^{15f}R^{16f}, wherein R^{15f} and R^{16f} independently represent hydrogen or C₁₋₆ alkyl or together form a heterocyclic ring;
Z^{f} represents CO or SO₂;
R² represents halogen, C₁₋₆ alkyl, C₁₋₆ alkoxy, cyano, amino or trifluoromethyl;
n is 0, 1 or 2;
R³ represents —(CH₂)_{q}—NR¹¹R¹² or a group of formula (i): 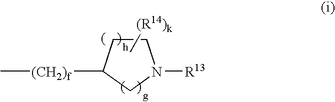
   wherein q is 2, 3 or 4;
R¹¹ and R¹² independently represent Con alkyl or together with the nitrogen atom to which they are attached represent an N-linked heterocyclic group selected from pyrrolidine, piperidine and homopiperidine optionally substituted by one or two R¹⁷ groups;
R¹³ represents C₁₋₆ alkyl, C₃₋₆cycloalkyl or -C₁₋₄alkyl-C₃₋₆cycloalkyl;
R¹⁴ and R¹⁷ independently represent halogen, C₁₋₆alkyl, haloC₁₋₆alkyl, OH, diC₁₋₆-alkylamino or C₁₋₆alkoxy;
f and k independently represent 0, 1 or 2;
g is 0, 1 or 2 and h is 0, 1, 2 or 3, such that g and h cannot both be 0; or solvates thereof.

In one particular aspect of the present invention, when R¹ represents a group of formula (F), R^{5f} is linked to Z^{f} via a carbon atom, u represents 1 and Z^{f} represents CO.

Alkyl groups, whether alone or as part of another group, may be straight chain or branched and the groups alkoxy and alkanoyl shall be interpreted similarly. Alkyl moieties are more preferably C₁₋₆ alkyl, eg. methyl or ethyl. The term ‘halogen’ is used herein to describe, unless otherwise stated, a group selected from fluorine, chlorine, bromine or iodine.

The term “aryl” includes single and fused rings wherein at least one ring is aromatic, for example, phenyl, naphthyl and tetrahydronaphthalenyl.

The term “heterocyclyl” is intended to mean a 4-7 membered monocyclic saturated or partially unsaturated aliphatic ring or a 4-7 membered monocyclic saturated or partially unsaturated aliphatic ring fused to a benzene ring containing 1 to 3 heteroatoms selected from oxygen or nitrogen. Suitable examples of such monocyclic rings include pyrrolidinyl, piperidinyl, piperazinyl, morpholinyl, diazepanyl, azepanyl, dihydroimidazolyl, tetrahydropyranyl and tetrahydrofuranyl. Suitable examples of benzofused heterocyclic rings include indolinyl, isoindolinyl and tetrahydroisoquinolinyl.

The term “nitrogen containing heterocyclyl” is intended to represent any heterocyclyl group as defined above which contains a nitrogen atom.

The term “heteroaryl” is intended to mean a 5-7 membered monocyclic aromatic or a fused 8-11 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like.

Preferably, n represents 0.

Preferably, R³ represents —(CH₂)_{q}—NR¹¹R¹².

Preferably, q is 3.

Preferably, NR¹¹R¹² represents an N-linked heterocyclic group, more preferably unsubstituted piperidine.

For compounds of formula (I) wherein R¹ represents a group of formula (A):

Preferably, R^{5a} represents:
hydrogen;
C₁₋₆ alkyl (eg. methyl or i-propyl) optionally substituted by —CONR^{15a}R^{16a} (eg. CONMe₂, CONMe-phenyl, CO—N-piperidine or CO—N-pyrrolidine);
C₁₋₆ alkoxycarbonyl (eg. t-butoxycarbonyl);
aryl (eg. phenyl) optionally substituted by one or more (eg. 1, 2 or 3) cyano, halogen (eg. fluorine or chlorine), C₁₋₆alkyl (eg. methyl), C₁₋₆alkoxy (eg. methoxy), polyhaloC₁₋₆alkyl (eg. trifluoromethyl) or C₁₋₆ alkanoyl (eg. COCH₃) groups;
heteroaryl (eg. pyridyl, pyrimidyl, pyridazinyl, pyrazinyl, quinolinyl or benzothiazolyl) optionally substituted by one or more (eg. 1, 2 or 3) oxo, cyano, halogen (eg. chlorine), C, e alkyl (eg. methyl) or polyhaloC₁₋₆ alkyl (eg. trifluoromethyl) groups;
C₁₋₆ alkyl-heterocyclyl (eg. —CH₂-tetrahydrofuranyl);
C₃₋₈cycloalkyl (eg. cycloheptyl);
—C₁₋₆alkyl-heteroaryl (eg. —CH₂-pyridyl);
heteroaryl-aryl (eg. -thiadiazolyl-phenyl); or
—CH(aryl)(aryl) (eg. —CH(phenyl)(phenyl)).

Preferably, m represents 1.

When n represents 1, R² is preferably halogen (eg. fluorine) or trifluoromethyl. When n represents 2, R² is preferably halogen (eg. fluorine).

Preferably, p represents 0, 1 or 2, more preferably 0.

When p represents 1, preferably R^{4a} represents oxo or C₁₋₆ alkyl (eg. methyl).

When p represents 2, preferably R^{4a} represents C₁₋₆alkyl (eg. methyl) or forms a methylene bridging group.

For compounds of formula (I) wherein R¹ represents a group of formula (B):

Preferably, NR^{4b}R^{5b} represents an N-linked heterocyclyl (eg. morpholinyl, piperidinyl, indolinyl, isoindolinyl or piperazinyl) or a -heterocyclyl_X^{b}-aryl group (eg. -piperidinyl-phenyl, -piperazinyl-phenyl, -piperazinyl-CO-phenyl or -piperazinyl-CO-naphthyl) optionally substituted by a polyhaloC₁₋₆alkoxy (eg. trifluoromethoxy) group.

For compounds of formula (I) wherein R¹ represents a group of formula (C): When present, R^{4c} preferably represents aryl (eg. phenyl), C₁₋₆ alkyl (eg. methyl), OH or an optionally substituted heteroaryl group (eg. dihydroimidazol-2-one substituted by phenyl), more preferably R^{4c} represents methyl.

When n represents 1, R² is preferably halogen (eg. fluorine) or trifluoromethyl. When n represents 2, R² is preferably halogen (eg. fluorine).

When r represents 2, preferably R^{4c} represents methyl.

For compounds of formula (I) wherein R¹ represents a group of formula (D): Preferably, R^{4d} represents phenyl or naphthyl, more preferably unsubstituted phenyl or naphthyl.

For compounds of formula (I) wherein R¹ represents a group of formula (E^{a}):
X^{e} is preferably O or NH, R^{4e} is preferably aryl (eg. phenyl) or -C₁₋₆ alkyl-aryl (eg. benzyl) and Y^{e} is preferably —CH₂—.

For compounds of formula (I) wherein R¹ represents a group of formula (E^{b}):
R^{5e} is preferably aryl (eg. phenyl).

For compounds of formula (I) wherein R¹ represents a group of formula (E^{c}):
R^{6e} and R^{7e}, together with the carbon atoms to which they are attached preferably form a benzene ring and [symbol] is preferably a double bond.

For compounds of formula (I) wherein R¹ represents a group of formula (F):

Preferably, R⁵′ represents:

C₁₋₆alkyl (eg. i-propyl);
C₃₋₈cycloalkyl (eg. cyclohexyl or cycloheptyl);
aryl (eg. phenyl or tetrahydronaphthalene) optionally substituted by a halogen atom (eg. chlorine), cyano, N-propyl₂SO₂— or a polyhaloC₁₋₆ alkyl group (eg. trifluoromethyl);
heteroaryl (eg. furyl, thienyl, pyridyl, quinoxaline, pyrazine, 1,2,3-benzothiadiazole, benzofuranyl, isoxazole or pyrazole) optionally substituted by a halogen atom (eg. chlorine), polyhaloC₁₋₆ alkyl group (eg. trifluoromethyl) or C₁₋₆ alkyl (eg. methyl or t-butyl);
heterocyclyl (eg. morpholine, pyrrolidine, tetrahydrofuran or tetrahydropyran);
C₁₋₆ alkyl-aryl (eg. α-methylbenzyl or α,α-dimethylbenzyl).

Preferably, R^{5f} is optionally substituted by one or more (eg. 1, 2 or 3) halogen (eg. chlorine), cyano, trifluoromethyl, C₁₋₆alkyl (eg. methyl or t-butyl), MeSO₂— or N-propyl₂SO₂ groups.

More preferably, R^{5f} represents C₃₋₈ cycloalkyl (eg. cyclohexyl), heteroaryl (eg. furyl) or aryl (eg. phenyl or tetrahydronaphthalene) optionally substituted by a cyano group.

Preferably, Z^{f} represents CO.

When n represents 1, R² is preferably trifluoromethyl.

Preferably, t represents 0 or 2, more preferably 0.

When t represents 2, both R^{4f} groups are preferably methyl or form a methylene bridging group.

Preferably, u represents 1.

When R³ represents a group of formula (i), preferably f represents 0, h represents 1, g represents 2, k represents 0 and R¹³ represents C₁₋₆alkyl (eg. isopropyl) or C₃₋₆ cycloalkyl (eg. cyclobutyl or cyclopentyl).

Preferred compounds according to the invention include examples E1-E172 as shown below, or a pharmaceutically acceptable salt thereof.

Compounds of formula (I) may form acid addition salts with acids, such as conventional pharmaceutically acceptable acids, for example maleic, hydrochloric, hydrobromic, phosphoric, acetic, fumaric, salicylic, sulphate, citric, lactic, mandelic, tartaric and methanesulphonic. Salts, solvates and hydrates of histamine H3 receptorantagonists therefore form an aspect of the invention.

Certain compounds of formula (I) are capable of existing in stereoisomeric forms. It will be understood that the invention encompasses all geometric and optical isomers of these compounds and the mixtures thereof including racemates. Tautomers also form an aspect of the invention.

The present invention also provides a process for the preparation of a compound of formula (I) or a pharmaceutically acceptable salt thereof, which process comprises:
(a) preparing a compound of formula (I) wherein R¹ represents a group of formula (A) which comprises reacting a compound of formula (II) 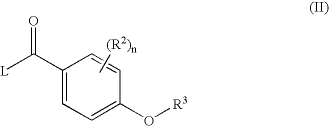
   with a compound of formula (III) 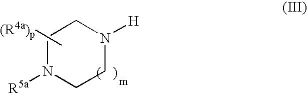
   or a protected derivative thereof, wherein R², R³, R^{4a}, R^{5a}, m, n and p are as defined above and L is OH or a suitable leaving group (eg. a halogen atom such as chlorine); or
(b) preparing a compound of formula (I) wherein R¹ represents a group of formula (A) and wherein R³ represents —(CH₂)_{q}—NR¹¹R¹² which comprises reacting a compound of formula (IV) 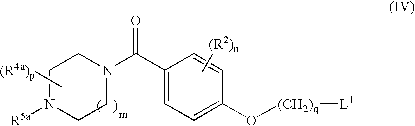
   wherein R², R^{4a}, R^{5a}, m, n, p and q are as defined above and L¹ represents a suitable leaving group such as a halogen atom (eg. bromine) with a compound of formula HNR¹¹R¹²; wherein R¹¹ and R¹² are as defined above; or
(c) preparing a compound of formula (1) wherein R¹ represents a group of formula (B) which comprises reacting a compound of formula (V) 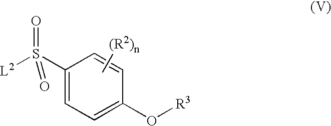
   with a compound of formula R^{4b}R^{5b}NH wherein R², R³, R^{4b}, R^{5b} and n are as defined above and L² is OH or a suitable leaving group (eg. a halogen atom such as chlorine); or
(d) preparing a compound of formula (I) wherein R¹ represents a group of formula (B) and wherein R³ represents —(CH₂)_{q}—NR¹¹R¹² which comprises reacting a compound of formula (VI) 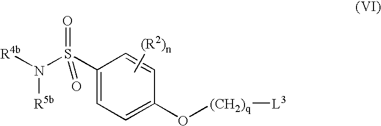
   wherein R², R^{4b}, R^{5b}, n and q are as defined above and L³ represents a suitable leaving group such as a halogen atom (eg. bromine) with a compound of formula HNR¹¹R¹²; wherein R¹¹ and R¹² are as defined above; or
(e) preparing a compound of formula (I) wherein R¹ represents a group of formula (B) which comprises reacting a compound of formula (VII) 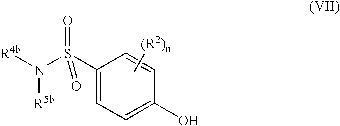
   wherein R², R^{4b}, R^{5b} and n are as defined above, with a compound of formula R³-L⁴, wherein R³ is as defined above and L⁴ represents a suitable leaving group such as a halogen atom or an OH group; or
(f) preparing a compound of formula (I) wherein R¹ represents a group of formula (C) which comprises reacting a compound of formula (II) as defined above, with a compound of formula (VIII) 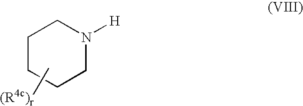
   or a protected derivative thereof, wherein R^{4c} and r are as defined above; or
(g) preparing a compound of formula (I) wherein R¹ represents a group of formula (C) and wherein R³ represents —(CH₂)_{q}—NR¹¹R¹² which comprises reacting a compound of formula (IX) 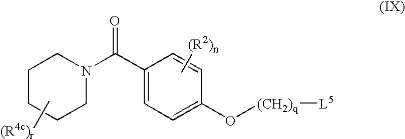
   wherein R², n, R^{4c}, r, and q are as defined above and L⁵ represents a suitable leaving group such as a halogen atom (eg. bromine) with a compound of formula HNR¹¹R¹²; wherein R¹¹ and R¹² are as defined above; or
(h) preparing a compound of formula (I) wherein R¹ represents a group of formula (D) which comprises reacting a compound of formula (II) as defined above, with a compound of formula (X) 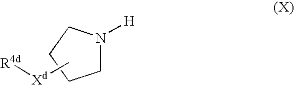
   or a protected derivative thereof, wherein R^{4d} and X^{d} are as defined above; or
(i) preparing a compound of formula (I) wherein R¹ represents a group of formula (D) and wherein R³ represents —(CH₂)_{q}—NR¹¹R¹² which comprises reacting a compound of formula (XI) 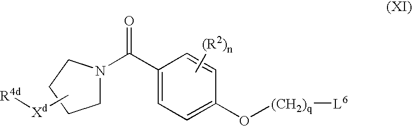
   wherein R^{4d}, X^{d}, R², n, and q are as defined above and L⁶ represents a suitable leaving group such as a halogen atom (eg. bromine) with a compound of formula HNR¹¹R¹²; wherein R¹¹ and R¹² are as defined above; or
(j) preparing a compound of formula (I) wherein R¹ represents a group of formula —CO-E^{a}, —CO-E^{b} or —CO-E^{c} which comprises reacting a compound of formula (II) as defined above, with a compound of formula H-E^{a}, H-E^{b} or H-E^{c} or a protected derivative thereof, wherein E^{a}, E^{b} and E^{c} are as defined above; or
   (k) preparing a compound of formula (I) wherein R¹ represents a group of formula —CO-E and wherein R³ represents —(CH₂)_{q}—NR¹¹R¹² which comprises reacting a compound of formula (XII) 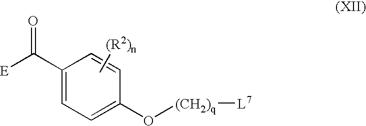
      wherein R², n, q and E are as defined above and L⁷ represents a suitable leaving group such as a halogen atom (eg. bromine) with a compound of formula HNR¹¹R¹²; wherein R¹¹ and R¹² are as defined above; or
(l) preparing a compound of formula (I) wherein R¹ represents a group of formula (F) which comprises reacting a compound of formula (II) as defined above, with a compound of formula (XIII) 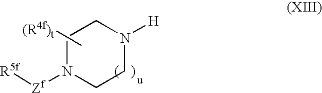
   or a protected derivative thereof, wherein R^{5f}, Z^{f}, R^{4f}, u and t are as defined above; or
(m) preparing a compound of formula (I) wherein R¹ represents a group of formula (F) and wherein R³ represents —(CH₂)_{q}—NR¹¹R¹² which comprises reacting a compound of formula (XIV) 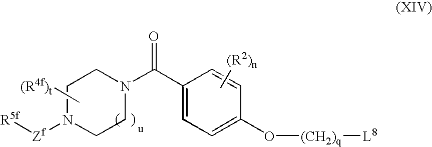
   wherein R^{5f}, Z^{f}, R², R^{4f}, n, t, u and q are as defined above and L⁸ represents a suitable leaving group such as a halogen atom (eg. bromine) with a compound of formula HNR^{11a}R^{12a}; wherein R^{11a} and R^{12a} are as defined above for R¹¹ and R¹² or a group convertible thereto; or
(n) preparing a compound of formula (I) wherein R¹ represents a group of formula (F) which comprises reacting a compound of formula (XV) 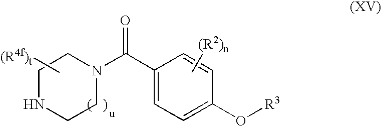
   or a protected derivative thereof, wherein R², R³, R^{4f}, n, t and u are as defined above, with a compound of formula R^{5fa}-Z^{f}-L⁹, wherein R^{5fa} is as defined above for R^{5f} or a group convertible thereto, Z^{f} is as defined above and L⁹ represents a suitable leaving group, such as a halogen atom (eg. chlorine) or a hydroxy group which may be converted into a suitable leaving group; and optionally thereafter
(o) deprotecting a compound of formula (I) which is protected; and optionally thereafter
(p) interconversion to other compounds of formula (I).

Process (a) typically comprises halogenation of the compound of formula (II) with a suitable halogenating agent (eg. thionyl chloride) followed by reaction with the compound of formula (III) in the presence of a suitable base such as triethylamine or a solid supported amine, in a suitable solvent such as dichloromethane. Process (a) may also typically comprise activation of the compound of formula (II) with a coupling reagent such as dicyclohexylcarbodiimide or solid supported carbodiimide in a suitable solvent such as N,N-dimethylfonmamide followed by reaction with the compound of formula (III).

Processes (b), (d), (g), (i), (k) and (m) are typically performed in the presence of a suitable solvent (such as 1-butanol) at an elevated temperature.

Process (c) typically comprises reaction with the compound of formula R^{4b}R^{5b}NH optionally in the presence of a suitable base such as triethylamine or a solid supported amine, in a suitable solvent such as dichloromethane. When L² represents OH, process (c) typically comprises an initial halogenation reaction of the compound of formula (V) with a suitable halogenating agent (eg. thionyl chloride) prior to reaction with the compound of formula R^{4b}R^{5b}NH as above.

Process (e) typically comprises an alkylation reaction under Mitsunobu conditions.

Processes (f), (h), (j) and (l) typically comprise reaction with the compound of formula (VIII), (X), H-E^{a}, H-E^{b}, H-E^{c} or (XIII) optionally in the presence of a suitable base such as triethylamine or a solid supported amine, in a suitable solvent such as dichloromethane. When L represents OH, processes (f), (h), (j) and (l) typically comprise an initial halogenation reaction of the compound of formula (II) with a suitable halogenating agent (eg. thionyl chloride) prior to reaction with the compound of formula (VIII), (X), H-E^{a}, H-E^{b}, H-E^{c} or (XIII) as above.

When L represents OH, processes (f), (h), (j) and (l) may also typically comprise activation of the compound of formula (II) with a coupling reagent such as dicyclohexylcarbodiimide or solid supported carbodiimide in a suitable solvent such as N,N-dimethylformamide followed by reaction with the compound of formula (VIII), (X), H-E^{a}, H-E^{b}, H-E^{c} or (XIII).

Process (n) typically comprises the use of a suitable base, such as triethylamine or a solid supported base such as diethylaminomethylpolystyrene in a suitable solvent such as dichloromethane. Process (n) may also involve activation of a carboxylic acid with a suitable coupling agent such as dicyclohexylcarbodiimide followed by reaction with the compound of formula (XV).

In process (o), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Process (p) may be performed using conventional interconversion procedures such as epimerisation, oxidation, reduction, alkylation, nucleophilic or electrophilic aromatic substitution, ester hydrolysis or amide bond formation.

Compounds of formula (II) wherein R³ represents —(CH₂)_{q}—NR¹¹R¹² may be prepared in accordance with the following procedure: 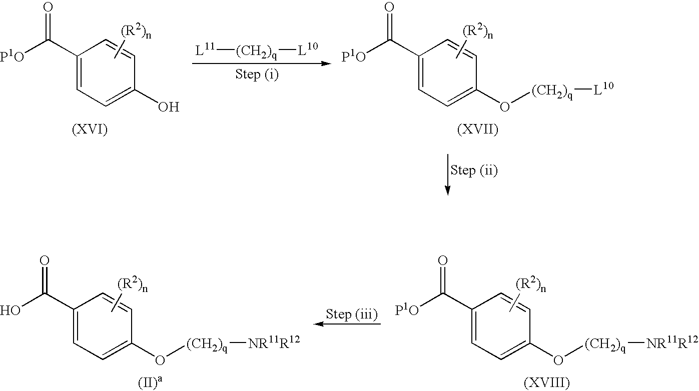
wherein R², n, q, R¹¹ and R¹² are as defined above, P¹ represents a protecting group such as methyl, ethyl or t-butyl, L¹⁰ and L¹¹ independently represent a leaving group such as halogen (eg. L¹⁰ represents chlorine and L¹¹ represents bromine). The —CO₂H group of compounds of formula (II)^{a} may be converted to —COL wherein L represents a leaving group by, for example, halogenation using thionyl chloride.

Step (i) typically comprises reaction of a compound of formula (XVI) with a suitable alkylating agent such as 1-bromo-3-chloropropane in a suitable solvent such as acetone in the presence of potassium carbonate.

Step (ii) typically comprises treatment of a compound of formula (XVII) with an amine of formula HNR¹¹R¹².

Step (iii) comprises a deprotection reaction which may be performed for example under acidic conditions with hydrochloric acid.

Compounds of formula (IV) or (XIV) may be prepared by hydrolysing a compound of formula (XVII) as defined above under suitable conditions (eg. under acidic conditions with HCl), suitably activated (eg. by conversion into the acid chloride with thionyl chloride), followed by treatment with a compound of formula (III) or (XIII), respectively as defined above.

Compounds of formula (II) wherein R³ represents —(CH₂)_{q}—NR¹¹R¹² may also be prepared in accordance with the following procedure: 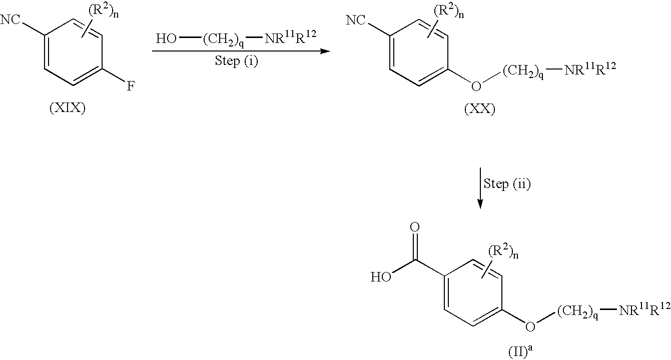
wherein R², n, q, R¹¹ and R¹² are as defined above.

Step (i) typically comprises reaction of a compound of formula (XIX) in the presence of a suitable base such as sodium hydride in an appropriate solvent such as dimethylsulfoxide or N,N-dimethylformamide.

Step (ii) typically comprises a hydrolysis reaction for example under acidic conditions using hydrochloric acid.

Compounds of formula (IV), (IX), (XI), (XII) and (XIV) may be prepared using an analogous procedure using HO—(CH₂)_{q}—L¹², wherein q is as defined above and L¹² represents an OH group or a group convertible to a leaving group.

Compounds of formula (II) wherein R³ represents a group of formula (i) may be prepared in a similar manner to the procedure shown above.

Compounds of formula (V) wherein L² represents chlorine may be prepared in accordance with the following procedure: 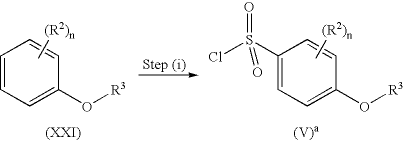
wherein R², R³ and n are as defined above.

Step (i) typically comprises reaction of a compound of formula (XXI) with a suitable reagent such as chlorosulfonic acid in a suitable solvent such as chloroform.

Compounds of formula (VI) may be prepared in accordance with the following procedure: 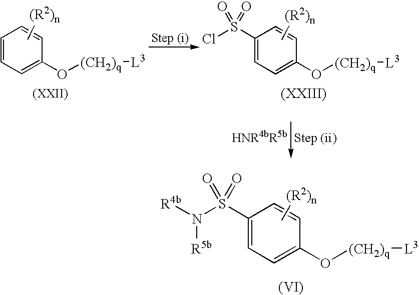
wherein R², n, q, L³, R^{4b} and R^{5b} are as defined above.

Step (i) may be performed by reacting a compound of formula (XXII) with a suitable reagent such as chlorosulfonic acid in a suitable solvent such as chloroform.

Step (ii) is typically performed in the presence of a suitable solvent such as dichloromethane.

Compounds of formula (VII) may be prepared in accordance with the following procedure: 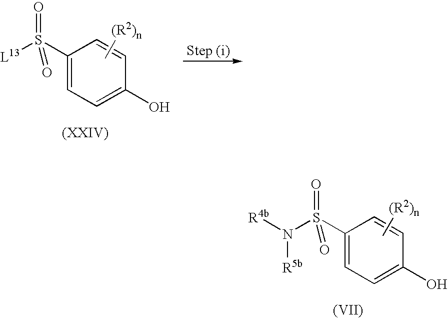
wherein R^{4b}, R^{5b}, R² and n are as defined above and L¹³ represents a suitable leaving group such as a halogen atom (eg. chlorine).

Step (i) typically comprises reaction of a compound of formula (XXIV) with a compound of formula R^{4b}R^{5b}NH, wherein R^{4b} and R^{5b} are as defined above, in a suitable solvent such as dichloromethane.

Compounds of formula (VIII) are either commercially available or may be prepared via standard routes, for example, imidazolones (e.g. piperidin-4-yl-4-phenyl-1,3-dihydroimidazol-2-one) may be prepared using the procedures described by Carling et al., J. Med. Chem., 1999, 42, 2706.

Compounds of formula (XV) may be prepared in accordance with the following procedure: 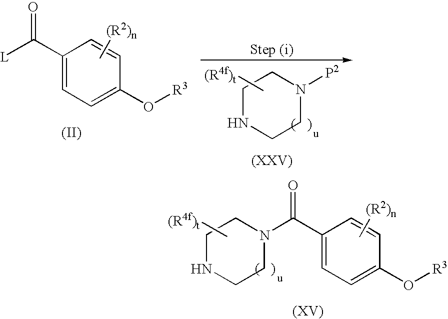
wherein L, R², n, R³, R⁴′, t and u are as defined above and p² represents a suitable protecting group such as t-butoxycarbonyl (t-Boc) or t-butyl.

Compounds of formula H-E^{a}, H-E^{b} and H-E^{c} are either commercially available or may be prepared via standard routes, for example, spiro imidazolones (e.g 3-benzyl-2-oxo-1,3,8-triazaspiro[4.5]decane) can be prepared as described by Smith et al., J. Med. Chem., 1995, 38, 3772, spiro morpholinones (e.g. 1-oxa-4,9-diazaspiro[5.5]undecan-3-one) may be prepared as described by Clark et al., J. Med. Chem., 1983, 26, 855, spiro oxazolidinones (e.g. 3-phenyl-1-oxa-3,8-diazaspiro[4.5]decan-2-one) may be prepared as described by Caroon et al., J. Med. Chem., 1981, 24, 1320.

Compounds of formula R^{4b}R^{5b}NH, (III), (X), (XIII), (XVI), (XIX), (XXI), (XXII), (XXIV) and (XXV) are either known in the literature or can be prepared by analogous methods.

Compounds of formula (I) and their pharmaceutically acceptable salts have affinity for and are antagonists and/or inverse agonists of the histamine H3 receptor and are believed to be of potential use in the treatment of neurological diseases including Alzheimer's disease, dementia, age-related memory dysfunction, mild cognitive impairment, cognitive deficit, epilepsy, neuropathic pain, inflammatory pain, migraine, Parkinson's disease, multiple sclerosis, stroke and sleep disorders including narcolepsy; psychiatric disorders including schizophrenia (particularly cognitive deficit of schizophrenia), attention deficit hypereactivity disorder, depression and addiction; and other diseases including obesity, asthma, allergic rhinitis, nasal congestion, chronic obstructive pulmonary disease and gastrointestinal disorders.

Thus the invention also provides a compound of formula (I) or a pharmaceutically acceptable salt thereof, for use as a therapeutic substance in the treatment or prophylaxis of the above disorders, in particular cognitive impairments in diseases such as Alzheimer's disease and related neurodegenerative disorders.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment of the above disorders.

When used in therapy, the compounds of formula (I) are usually formulated in a standard pharmaceutical composition. Such compositions can be prepared using standard procedures.

Thus, the present invention further provides a pharmaceutical composition for use in the treatment of the above disorders which comprises the compound of formula (I) or a pharmaceutically acceptable salt thereof and a pharmaceutically acceptable carrier.

The present invention further provides a pharmaceutical composition which comprises the compound of formula (I) or a pharmaceutically acceptable salt thereof and a pharmaceutically acceptable carrier.

Compounds of formula (I) may be used in combination with other therapeutic agents, for example histamine H1 antagonists or medicaments claimed to be useful as either disease modifying or symptomatic treatments of Alzheimer's disease. Suitable examples of such other therapeutic agents may be agents known to modify cholinergic transmission such as 5-HT₆ antagonists, M1 muscarinic agonists, M2 muscarinic antagonists or acetylcholinesterase inhibitors. When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three a day. Such therapy may extend for a number of weeks or months.

The following Descriptions and Examples illustrate the preparation of compounds of the invention.

Description 1

### Ethyl 4-(3-Piperidin-1-ylpropoxy)benzoate (D1)

A stirred mixture of ethyl 4-(3-chloropropoxy)benzoate (4.73 g) (D. A. Walsh et al J. Med. Chem. 1989, 32(1), 105), piperidine (2.9 ml), sodium carbonate (3.1 g) and potassium iodide (162 mg) in 1-butanol (50 ml) was heated at 105° C. for 16 h. The reaction was cooled to rt, diluted with EtOAc (100 ml), washed with water (3×50 ml), saturated brine (50 ml), dried (MgSO₄) and evaporated to give the title compound (D1) (6.88 g). MS electrospray (+ ion) 292 (MH⁺). ¹H NMR δ (CDCl₃): 7.98 (2H, d, J=8.8 Hz), 6.90 (2H, d, J=8.8 Hz), 4.34 (2H, q ,J=7.5 Hz), 4.06 (2H, t, J=6.3 Hz), 2.46 (4H, m), 2.00 (2H, m), 1.50 (6H, m), 1.38 (3H, t, J=7.5 Hz).

Description 2

### 4-(3-Piperidin-1-ylpropoxy)benzoic acid hydrochloride (D2)

A solution of ethyl 4-(3-piperidin-1-ylpropoxy)benzoate (D1) (1.4 g) in concentrated hydrochloric acid (15 ml) was heated under reflux for 1 h, cooled and evaporated to give the title compound (D2) (1.029). MS electrospray (+ ion) 264 (MH⁺).¹H NMR δ (DMSO-d6): 10.59 (1H, s), 10.25 (1H, s), 7.90 (2H, d, J=9 Hz), 7.02 (2H, d, J=9 Hz), 4.14 (2H, t, J=6 Hz), 3.05-3.52 (4H, m), 2.91 (2H, m), 2.20 (2H, m), 1.25-1.91 (6H, m).

Description 3

### 4-(3-Piperidin-1-ylpropoxy)benzoyl chloride hydrochloride (D3)

4-(3-Piperidin-1-ylpropoxy)benzoic acid hydrochloride (D2) (0.23 g) in thionyl chloride (5 ml) was heated under reflux for 1 h. The reaction mixture was then evaporated to a minimum and coevaporated from DCM (3×10 ml) to give the title compound (D3) as a white powder (0.24 g).

Description 4

### 1-[4-(3-Piperidin-1-ylpropoxy)benzoyl]-4-t-butoxycarbonylpiperazine (D4)

To t-butoxycarbonylpiperazine (5.65 g) in DCM (70 ml) was added triethylamine (16.2 ml) followed by slow addition of 4-(3-piperidin-1-ylpropoxy)benzoyl chloride hydrochloride (D3) (10.60 g) in DCM (100 ml). The reaction was stirred at rt for 3 h, then washed with saturated sodium hydrogen carbonate solution (2×200 ml) followed by brine (100 ml). The organic layer was dried (MgSO₄) and evaporated to a brown solid which was purified by chromatography [silica gel; 0-6% MeOH (containing 10% 0.880 ammonia solution)/DCM] to give the title compound (D4) as a pale brown solid (12.05 g).

Description 5

### 1-[4-(3-Piperidin-1-ylpropoxy)benzoyl]piperazine Dihydrochloride (D5)

To 1-[4-(3-piperidin-1-ylpropoxy)benzoyl]4-t-butoxycarbonylpiperazine (D4) (12.05 g) in DCM (150 ml) was added 4N HCl/Dioxane (35 ml), forming a white precipitate. The reaction was stirred for 2.5 hours before evaporation. The white crude solid was triturated with DCM and dried overnight at 50° C. to yield the title compound (D5) (8.26 g).

Description 6

### 1-[4-(3-Piperidin-1-ylpropoxy)benzoyl]-4-t-butoxycarbonylhomopiperazine (D6)

Description 6 was prepared in accordance with the procedure described for Example 172.

Description 7

### 1-[4-(3-Piperidin-1-ylpropoxy)benzoyl]homopiperazine dihydrochloride (D7)

To 1-[4-(3-piperidin-1-ylpropoxy)benzoyl]4-t-butoxycarbonylhomopiperazine (D6) (1.50 g) in DCM (20 ml) was added 4N HCl (4 ml) and the mixture was allowed to stir at rt overnight. Evaporation of solvent followed by drying under high vacuum afforded the title compound (D7) as a white solid (1.5 g).

Description 8

### (1S,4S)-5-[4-(3-Piperidin-1-ylpropoxy)benzoyl]-2,5-diaza-bicyclo[2.2.1]heptane-2Carboxylic Acid t-Butyl Ester (D8)

Description 8 was prepared in accordance with the procedure described for Example 103.

Description 9

### (1S,4S)-2-[4-(3-Piperidin-1-ylpropoxy)benzoyl]-2,5-diaza-bicyclo[2.2.1]heptane dihydrochloride (D9)

Description 9 was prepared in accordance with the procedure described for Example 104.

Description 10

### (3R,5S)-1-[4-(3-Piperidin-1-ylpropoxy)benzoyl]-3,5-dimethylpiperazine (D10)

(2R,6S)-2,6-Dimethyl-piperazine (0.4 g) was dissolved in THF (30 ml) and treated with n-butyl lithium (1.6M solution in hexanes, 4.82 ml) under argon. The mixture was stirred at rt for 30 min and then 4-(3-piperidin-1-ylpropoxy)benzoyl chloride hydrochloride (D3) (1.0 g), dissolved in DCM (10 ml), was added dropwise. The reaction was stirred for 1 h and then evaporated to a minimum and the crude residue purified by column chromatography [silica gel, eluted with 0-10% MeOH (containing 10% 0.880 ammonia solution) in DCM] to afford the title compound (D10) as a yellow oil (0.65 g).

Description 11

### (S)-N-[4-(3-Piperidin-1-ylpropoxy)benzoyl]-3-aminopyrrolidine dihydrochloride (D11)

A solution of 4-(3-piperidin-1-ylpropoxy)benzoic acid hydrochloride (D2) (515 mg) in thionyl chloride (10 ml) was refluxed for 1 h, cooled to rt and evaporated. The acid chloride was re-evaporated from DCM (2×10 ml). The residue was redissolved in DCM (5 ml) and triethylamine (0.67 ml) and added to an ice cold stirred solution of (S)-3-t-butoxycarbonylaminopyrrolidine (304 mg) The solution was allowed to gain rt, stirred for 1 h. and then chromatographed (silica gel, step gradient 2-6% MeOH in DCM). Fractions containing the required product were treated with excess hydrogen chloride (4M solution in dioxan) for 2 h and then concentrated to yield the title compound (D11) (650 mg). MS electrospray (+ ion) 332 (MH⁺). ¹H NMR δ (DMSO-d₆), 10.38 (1H, s), 8.40 (3H, s), 7.52 (2H, d, J=9 Hz), 6.99 (2H, d, J=9 Hz), 4.11 (2H, t, 6 Hz), 2.75-3.92 (11H, m), 2.85 (2H, m), 1.90-2.30 (4H, m), 1.38-1.88 (6H, m).

Description 12

### 1-Bromo-3-(4-chlorosulfonylphenoxy)propane (D12)

A stirred solution of 3-bromo-1-phenoxypropane (4.3 g) in chloroform (20 ml) at −5° C. was treated dropwise with a solution of chlorosulfonic acid (2.66 ml) in chloroform keeping the temperature below 0° C. The reaction was stirred for 5 min then allowed to gain rt and stirred for 4 days. The mixture was poured onto ice and allowed to gain rt. The organic layer was collected, washed with water (3×20 ml), saturated brine (20 ml), dried (MgSO₄) and evaporated to give the title compound (D12) (1.9 g). ¹H NMR δ (CDCl₃): 7.98 (2H, d, J=8.8 Hz), 7.05 (2H, d, J=8.8 Hz), 4.24 (2H, t, J=5.8 Hz), 3.61 (2H, t, J=5.8 Hz), 2.37 (2H, m).

Description 13

### 4-[4-(3-Bromopropoxy)benzenesulfonylimorpholine (D13)

A solution of 1-bromo-3-(4-chlorosulfonylphenoxy)propane (D12) (200 mg) in DCM (5 ml) was treated with morpholine (0.14 ml) and stirred for 1 h. The solution was chromatographed (silica, step gradient 15 to 30% EtOAc in light petroleum 40°-60°) to give the title compound (D13) (99 mg). MS electrospray (+ ion) 365 (MH⁺). ¹H NMR δ (CDCl₃): 7.69 (2H, d, J=9 Hz), 7.02 (2H, d, J=9 Hz), 4.19 (2H, t, J=5.8 Hz), 3.74 (4H, m), 3.61 (2H, t, J=5.8 Hz), 2.99 (4H, m), 2.36 (2H, m).

Description 14

### 4-(3-Piperidin-1-yl-propoxy)-2-trifluoromethyl-benzonitrile (D14)

4-Fluoro-2-trifluoromethyl-benzonitrile (1.20 g) was dissolved in THF (20 ml) and 3-piperidin-1-yl-propan-1-ol (0.91 ml) was added. The reaction was cooled to 0° C. and potassium hexamethyldisilazide (0.5M solution in toluene; 12.72 ml) was added dropwise. The reaction was stirred at rt overnight, then diluted with ethyl acetate (50 ml) and partitioned with aqueous 1 N HCl (50 ml). The aqueous layer was washed with ethyl acetate (50 ml), then basified to pH 8.0 with sodium hydrogen carbonate and extracted with ethyl acetate (3×75 ml). The combined organic extracts were dried (MgSO₄) and evaporated to give the title compound (D14) as a clear oil which crystallised on standing (0.80 g).

Description 15

### 4-(3-Piperidin-1-yl-propoxy)-2-trifluoromethyl-benzoic acid hydrochloride (D15)

4-(3-Piperidin-1-yl-propoxy)-2-trifluoromethyl-benzonitrile (D14) (0.80 g) was dissolved in conc. HCl (20 ml) and heated at 135° C. for 24 h. Concentrated sulfuric acid (10 ml) was added and the reaction heated at 135° C. for 36 h. The reaction mixture was then evaporated to a minimum and treated with 12.5 N sodium hydroxide solution until pH 12 was obtained. The mixture was filtered and the filtrate evaporated to a minimum. Conc. HCl was then added until pH 1. The mixture was evaporated and the solid residue was extracted several times with methanol. The combined extracts were evaporated to give the title compound (D15) as a white solid (0.90 g).

Description 16

### 4-(3-Piperidin-1-yl-propoxy)-2-trifluoromethyl-benzoyl chloride hydrochloride (D16)

4-(3-Piperidin-1-yl-propoxy)-2-trifluoromethyl-benzoic acid hydrochloride (D15) (0.9 g) was heated at reflux in thionyl chloride (20 ml) for 2 h. The reaction mixture was evaporated to a minimum then co-evaporated with DCM (3×) to give the title compound (D16) as a white solid (1.0 g)

Description 17

2,5-Difluoro-4-(3-piperidin-1-yl)propoxy)benzonitrile (D17)

The title compound was prepared using the method of Description 14 from 2,4,5-trifluorobenzonitrile.

Description 18

### 2,5-Difluoro-4-(3-piperidin-1-ylpropoxy)benzoic Acid Hydrochloride (D18)

2,5-Difluoro-4-(3-piperidin-1-ylpropoxy)benzonitrile (D17) (1.1 g) was dissolved in conc.

HCl and heated under reflux for 24 h. The reaction mixture was then cooled to 5° C. and the resultant precipitate filtered and dried at 50° C. under high vacuum to give the title compound (D18) (0.56 g).

Description 19

### 2,5-Difluoro-4-(3-piperidin-1-ylpropoxy)benzoyl chloride hydrochloride (D19)

The title compound was prepared from 2,5-difluoro-4-(3-piperidin-1-yl)propoxy)benzoic acid hydrochloride (D18) using the method of Description 16.

Description 20

### 2-Fluoro-4-(3-piperidin-1-ylpropoxy)benzonitrile (D20)

The title compound was prepared using the method of Description 14 from 2,4-difluorobenzonitrile.

Description 21

### 2-Fluoro-4-(3-piperidin-1-ylpropoxy)benzoic acid hydrochloride (D21)

2-Fluoro-4-(3-piperidin-1-ylpropoxy)benzonitrile (D20) (1.4 g) was dissolved conc. HCl and heated under reflux for 24 h. The reaction mixture was then cooled to 5° C. and the resultant precipitate filtered and dried at 50° C. under high vacuum to give the title compound (D21) (1.5 g).

Description 22

### 2-Fluoro-4-(3-piperidin-1-ylpropoxy)benzoyl chloride hydrochloride (D22)

The title compound was prepared from 2-fluoro-4-(3-piperidin-1-ylpropoxy) benzoic acid hydrochloride (D21) using the method of Description 16.

Description 23

### 1-tert-Butoxycarbonyl-4-[4-fluoro-2-trifluoromethyl-benzoyl]piperazine (D23)

4-Fluoro-2-(trifluoromethyl)benzoic acid (2.0 g) was dissolved in thionyl chloride (20 ml) and heated at reflux for 2 h. The reaction was then cooled and evaporated (co-evaporated with DCM×3) and then dissolved in DCM (50 ml). This solution was added slowly to 1-tert-butoxy-carbonylpiperazine (1.62 g), and TEA (2.54 ml), dissolved in DCM (50 ml). The reaction was then stirred at rt for 2 h before being washed with 1 N HCl (2×100 ml), saturated sodium hydrogen carbonate (2×100 ml) and brine (50 ml). The organic layer was dried (MgSO₄) and evaporated to give the title compound (D23) (3.09 g).

Description 24

### 1-tert-Butoxycarbonyl-4-[4-(3-piperidin-1-ylpropoxy)-2-trifluoromethyl-benzoyl]piperazine (D24)

1-tert-Butoxycarbonyl-4-[4-fluoro-2-trifluoromethyl-benzoyl]piperazine (D23) (2.05 g) and 3-(1-piperidinyl)-1-propanol (1.17 g) were dissolved in DMSO (30 ml) and KHMDS (12.2 ml, 20% in THF) was added slowly and the reaction was stirred for 30 min. The reaction mixture was then evaporated and re-dissolved in ethyl acetate and washed with saturated sodium hydrogen carbonate (2×80 ml) and brine (80 ml). The organic layer was dried (MgSO₄) and evaporated, and the residue purified by chromatography [silica gel; gradient elution with 0-10% MeOH (containing 10% 0.880 ammonia solution)/DCM].

Pure product fractions were evaporated and dried under high vacuum to give the title compound (D24) as a white solid (2.15 g).

Description 25

### 1-[4-(3-piperidin-1-ylpropoxy)-2-trifluoromethyl-benzoyl]piperazine dihydrochloride (D25)

1-tert-Butoxycarbonyl-4-[4-(3-piperidin-1-ylpropoxy)-2-trifluoromethyl-benzoyl]piperazine (D24) (2.15 g) was dissolved in DCM (50 ml) and 4N HCl in dioxane (25 ml) was added and the reaction stirred at rt overnight. The reaction mixture was then evaporated [co evaporated with toluene (3×), then acetone (3×)] to give the title compound (D25) as a white foam (1.82 g).

Description 26

### (3R,5S)-1-tert-Butoxycarbonyl-3,5-dimethyl-4-(4-fluorobenzoyl)piperazine dihydrochloride (D26)

(2R,6S)-2,6-Dimethylpiperazine (0.9 g) was stirred in THF (50 ml) and n-butyl lithium (2.5M in hexanes) (6.9 ml) was added. The mixture was stirred for 30 min and then TMSCI (1.1 ml) was added. The reaction was stirred for a further 30 min and then 4-fluorobenzoyl chloride (1.0 g) in THF (5 ml) was added dropwise and the reaction stirred for a further 30 min. Methanol (10 ml) was then added and the reaction evaporated to dryness. The crude amine intermediate was dissolved in DCM (30 ml) and TEA (1.23 ml) was added followed by di-tert-butyl dicarbonate (1.7 g) and the reaction stirred at rt under argon overnight. The mixture was then washed with saturated sodium hydrogen carbonate (3×50 ml) and brine (50 ml), dried (MgSO₄) and evaporated to yield the crude product which was purified by column chromatography [silica gel; gradient elution; 0-100% EtOAc:Hexane]. Fractions containing pure product were evaporated to give the title compound (D26) (0.67 g).

Description 27

### (3R,5S)-1-tert-Butoxycarbonyl-3,5-dimethyl-4-[4-(3-piperidin-1-yl)propoxybenzoyl]piperazine dihydrochloride (D27)

(3R,5S)1-tert-Butoxycarbonyl-3,5-dimethyl-4-(4-fluorobenzoyl)piperazine dihydrochloride (D26) (0.56 g) was dissolved in DMSO (5 ml) and 3-(1-piperidinyl)-1-propanol (0.249) was added followed by dropwise addition of KHMDS (0.5 M in toluene) (3.3 ml), and the reaction was stirred at rt under argon for 2 h. The reaction mixture was then evaporated and redissolved in ethyl acetate (100 ml), washed with saturated sodium hydrogen carbonate (3×50 ml), brine (50 ml) and dried (MgSO₄) before being evaporated. The crude product was chromatographed [silica gel, gradient elution, 0-10% MeOH (containing 10% 0.880 ammonia solution)/DCM]. Pure product fractions were evaporated to give the title compound (D27) as a clear oil (0.2 g).

Description 28

### (2R,6S)-2,6-Dimethyl-1-[4-(3-piperidin-1-yl)propoxybenzoyl]piperazine dihydrochloride (D28)

(3R,5S)-1-tert-Butoxycarbonyl-3,5-dimethyl-4-[4-(3-piperidin-1-yl)propoxybenzoyl]piperazine dihydrochloride (D27) (0.2 g) was dissolved in DCM (5 ml) and 4N HCl/dioxane (5 ml) was added and the reaction stirred for 16 h. The reaction mixture was then evaporated (co-evaporated with toluene 3×) to give the title compound (D28) as a white powder (0.18 g).

Description 29

### 4-[(1-tert-Butoxycarbonyl-4-piperidinyl)oxy]benzonitrile (D29)

4-Fluorobenzonitrile (3.0 g) was dissolved in THF (50 ml) and then N-tert-butoxy-carbonyl-4-piperidinol (4.98 g) was added. Potassium hexamethyldisilazide (20% wt solution in THF, 24.62 g) was then added dropwise and the reaction stirred at rt for 2 h. The reaction mixture was then evaporated to a minimum, redissolved in EtOAc (100 ml) and washed with aqueous 1N HCl (2×100 ml), saturated sodium bicarbonate solution (2×100 ml) and brine (100 ml). The organic layer was dried (MgSO₄) and then purified by chromatography [silica gel, step gradient 0-60% EtOAc/Hexane]. Fractions containing the required product were evaporated to give the title compound (D29) as a clear oil which crystallised on standing (6.83 g). ¹H NMR δ (CDCl₃): 7.59 (2H, d, J=7.50 Hz), 6.95 (2H, d, J=7.50 Hz), 4.44 (1H, m), 3.70 (2H, m), 3.38 (2H, m), 1.91 (2H, m), 1.77 (2H, m), 1.47 (9H, s).

Description 30

### 4-(4-Piperidinyloxy)benzonitrile trifluoroacetate (D30)

4-[(1-tert-Butoxycarbonyl-4-piperidinyl)oxy]benzonitrile (D29) (6.83 g) was dissolved in DCM (30 ml) and TFA (30 ml) was added. The reaction was stirred at rt for 1 h and then evaporated to give the title compound (D30) as a yellow oil (7.15 g—TFA salt plus 1.3 equivalents of TFA).

Description 31

### 4-[(1-Cyclobutyl-4-piperidinyl)oxy]benzonitrile (D31)

4-(4-Piperidinyloxy)benzonitrile trifluoroacetate (D30) (2.2 g) was dissolved in DCM (50 ml) and triethylamine (1.92 ml) was added followed by cyclobutanone (0.649). The mixture was stirred for 5 min, then sodium triacetoxyborohydride (1.94 g) was added and the reaction was stirred at rt under argon overnight. The reaction mixture was then washed with saturated potassium carbonate solution (3×30 ml) and brine (30 ml). The organic layer was dried (MgSO₄) and evaporated to give the title compound (D31) as a white solid (1.91 g). ¹H NMR δ (CDCl₃): 7.56 (2H, d, J=6.84 Hz), 6.93 (2H, d, J=6.80 Hz), 4.41 (1H, m), 2.77 (1H, m), 2.75 (2H, m), 2.30 (2H, m), 2.06 (4H, m), 1.87 (4H, m), 1.66 (2H, m).

Description 32

### 4-[(1-Isopropyl-4-piperidinyl)oxy]benzonitrile (D32)

The title compound was prepared in a similar manner to Description 31 using acetone in place of cyclobutanone.

Description 33

### 4-[(1-Cyclopentyl-4-piperidinyl)oxy]benzonitrile (D33)

The title compound was prepared in a similar manner to Description 31 using cyclopentanone in place of cyclobutanone.

Description 34

### 4-[(1-Cyclobutyl-4-piperidinyl)oxy]benzoic Acid Hydrochloride (D34)

4-[(1-Cyclobutyl-4-piperidinyl)oxy]benzonitrile (D31) (1.91 g) was dissolved in conc. HCl (30 ml) and heated to 120° C. for 2 h. The reaction mixture was then allowed to cool to rt and then further cooled to 5° C. The resultant white precipitate was filtered off and washed with a small quantity of water. The solid was then dried at 50° C. under vacuum overnight to yield the title compound (D34) as a white powder (0.95 g). ¹H NMR δ (DMSO-d₆): 12.60 (1H, s), 10.96 (1H, s), 7.90 (2H, d, J=8.70 Hz), 7.09 (2H, d, J=8.60 Hz), 4.09-4.64 (1H, m), 3.66-3.15 (3H, m), 2.99-2.77 (2H, m), 2.48-1.60 (10H, m).

Description 35

### 4-[(1-Cyclobutyl-4-piperidinyl)oxy]benzoyl chloride hydrochloride (D35)

4-[(1-Cyclobutyl-4-piperidinyl)oxy]benzoic acid hydrochloride (D34) (0.20 g) was dissolved in thionyl chloride (10 ml) and heated under reflux for 1.5 h. The thionyl chloride was removed by evaporation and the residue evaporated from DCM (3×10 ml) to give the title compound (D35) (0.21 g).

Description 36

### 4-[(1-Cyclobutyl-4-piperidinyl)oxy]benzoyl]4-t-butoxycarbonylpiperazine (D36)

To t-butoxycarbonylpiperazine (0.62 g) in DCM (50 ml) was added triethylamine (1.3 ml) followed by slow addition of 4-[(1-cyclobutylpiperidinyl)oxy]benzoyl chloride hydrochloride (D35) (1.16 g) in DCM (50 ml). The reaction was stirred at rt for 16 h, then washed with saturated sodium hydrogen carbonate solution (3×50 ml) followed by brine (50 ml). The organic layer was dried (MgSO₄) and evaporated to a brown solid which was purified by chromatography [silica gel; step gradient 0-10% MeOH (containing 10% 0.880 ammonia solution)/DCM] to give the title compound (D36) as a pale brown solid (1.0 g).

Description 37

### 4-[(1-Cyclobutyl-4-piperidinyl)oxy]benzoyl]piperazine dihydrochloride (D37)

To 4-[(1-cyclobutyl-4-piperidinyl)oxy]benzoyl]-4-t-butoxycarbonylpiperazine (D36) (1.0 g) in DCM (30 ml) was added 1N HCl in diethyl ether (30 ml), forming a white precipitate. The reaction was stirred for 16 h before evaporation. The white crude solid was dried overnight at 50° C. to yield the title compound (D37) (0.87 g).

Description 38

### 4-[(1-Isopropyl-4-piperidinyl)oxy]benzoyl]piperazine dihydrochloride (D38)

The title compound was prepared from 4-[(1-isopropyl-4-piperidinyl)oxy]benzonitrile (D32) following the procedures in Descriptions 34-37.

Description 39

### 4-[(1-Cyclopentyl-4-piperidinyl)oxy]benzoyl]piperazine dihydrochloride (D39)

The title compound was prepared from 4-[(1-cyclopentyl-4-piperidinyl)oxy]benzonitrile (D33) following the procedures in Descriptions 34-37.

### EXAMPLE 1

### N-[4-(3-Piperidin-1-ylpropoxy)benzoyl]-4-phenylpiperazine dihydrochloride (E1)

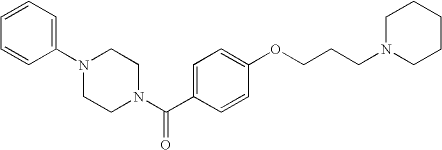

A solution of 4-(3-piperidin-1-ylpropoxy)benzoic acid hydrochloride (D2) (500 mg) in thionyl chloride (5 ml) was refluxed for 1 h, cooled to rt and evaporated. The acid chloride was re-evaporated from DCM (2×10 ml). The residue was redissolved in DCM (5 ml) and triethylamine (0.7 ml) and added to a stirred solution of 4-phenylpiperazine (270 mg) in DCM (20 ml) at rt. The mixture was stirred for 1 h, washed with saturated sodium hydrogen carbonate solution (10 ml), water (3×10 ml), dried (MgSO₄) and evaporated. The residue was chromatographed (silica gel, step gradient 2-6% MeOH in DCM). Fractions containing the required product were treated with excess hydrogen chloride (4M solution in dioxan) and then concentrated to yield the title compound (E1) (630 mg). MS electrospray (+ ion) 408 (MH⁺).¹H NMR δ (DMSO-d₆): 10.39 (1H,s), 6.90-7.47 (9H, m), 4.11 (2H, t, J=6 Hz), 2.66-3.89 (12H, m), 2.24 (2H, m), 1.22-1.83 (6H, m).

### EXAMPLE 2

### N-[4-(3-Piperidin-1-ylpropoxy)benzoyl]piperazine dihydrochloride (E2)

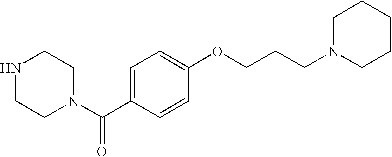

4-(3-Piperidin-1-ylpropoxy)benzoic acid hydrochloride (D2) (150 mg) was converted to the title compound (E2) by reaction with 4-t-butoxycarbonylpiperazine (93 mg) using the method described in Example 1 (E1) except that the treatment with excess hydrogen chloride (4M solution in dioxan) was continued for 2 h before evaporation (yield=125 mg). MS electrospray (+ ion) 332 (MH⁺).¹H NMR δ (DMSO-d6), 10.51 (1H, s), 9.50 (1H, s), 7.44 (2H, d, J=8.8 Hz), 7.00 (2H, d, J=8.8 Hz), 4.11 (2H, t, J=6 Hz), 3.71 (4H, m), 3.35 (8H, m), 2.87 (2H, m), 2.22 (2H, m), 1.30-1.90 (6H, m).

### EXAMPLES 3-5

### E3-5

Examples 3-5 were prepared from 4-(3-piperidin-1-ylpropoxy)benzoic acid hydrochloride (D2) and the appropriate amine using the method outlined in Example 1 (E1) and displayed ¹H NMR and mass spectral data that were consistent with structure.

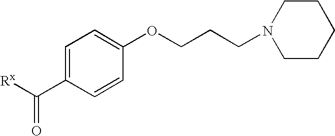

| | | Mass Spectrum |
|---|---|---|
| Example No | R^{x} | (ES⁺) |
| E3 | 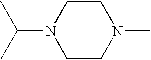 | 374 [M + H]⁺ |
| E4 | 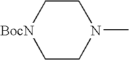 | 432 [M + H]⁺ |
| E5 | 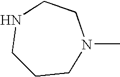 | 346 [M + H]⁺ |

### EXAMPLES 6-13

### E6-13

Examples 6-13 were prepared from 4-(3-piperidin-1-ylpropoxy)benzoic acid hydrochloride (D2) and the appropriate amine using the method outlined in Example 1 (E1) with the exception that polymer supported base was employed. All compounds displayed ¹H NMR and mass spectral data that were consistent with structure.

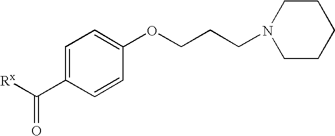

| Exam- | | |
|---|---|---|
| ple | | |
| No | R^{x} | Mass Spectrum |
| E6 | 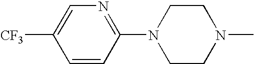 | 477 [M + H]⁺ |
| E7 | 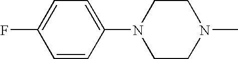 | 426 [M + H]⁺ |
| E8 | 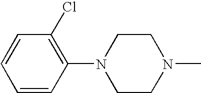 | 442, 444 [M + H]⁺ |
| E9 | 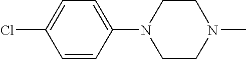 | 442, 444 [M + H]⁺ |
| E10 | 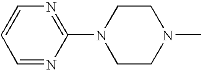 | 410 [M + H]⁺ |
| E11 | 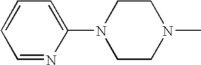 | 409 [M + H]⁺ |
| E12 | 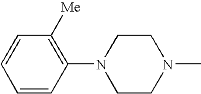 | 422 [M + H]⁺ |
| E13 | 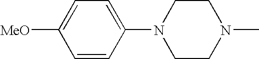 | 438 [M + H]⁺ |

### EXAMPLES 14-51

### E14-51

Examples 14-51 were prepared from 4-(3-piperidin-1-ylpropoxy)benzoic acid hydrochloride (D2) and the appropriate amine using the method outlined in Example 1 (E1) with the exception that diethylaminomethylpolystyrene was employed as the base. All compounds displayed ¹H NMR and mass spectral data that were consistent with structure.

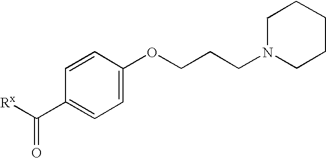

| Exam- | | |
|---|---|---|
| ple | | |
| No | R^{x} | Mass Spectrum |
| E14 | 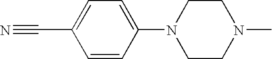 | 433 [M + H]⁺ |
| E15 | 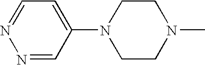 | 410 [M + H]⁺ |
| E16 | 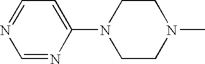 | 410 [M + H]⁺ |
| E17 | 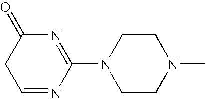 | 426 [M + H]⁺ |
| E18 | 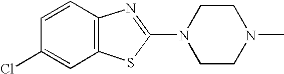 | 500/502 [M + H]⁺ |
| E19 | 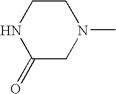 | 346 [M + H]⁺ |
| E20 | 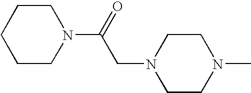 | 457 [M + H]⁺ |
| E21 | 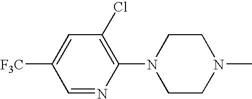 | 511/513 [M + H]⁺ |
| E22 | 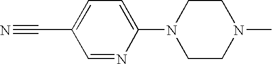 | 434 [M + H]⁺ |
| E23 | 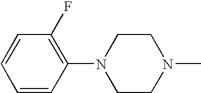 | 425 [M + H]⁺ |
| E24 | 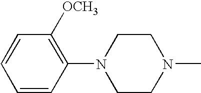 | 438 [M + H]⁺ |
| E25 | 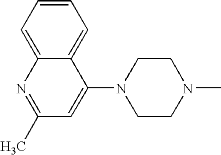 | 473 [M + H]⁺ |
| E26 | 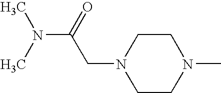 | 417 [M + H]⁺ |
| E27 | 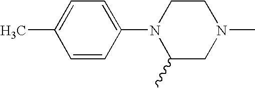 | 436 [M + H]⁺ |
| E28 | 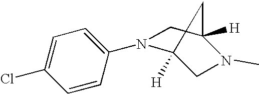 | 455/457 [M + H]⁺ |
| E29 | 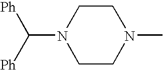 | 498 [M + H]⁺ |
| E30 | 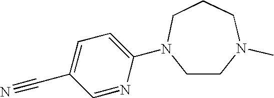 | 448 [M + H]⁺ |
| E31 | 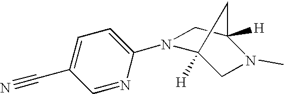 | 446 [M + H]⁺ |
| E32 | 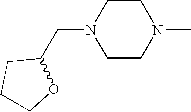 | 416 [M + H]⁺ |
| E33 | 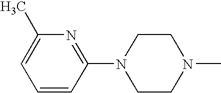 | 422 [M + H]⁺ |
| E34 | 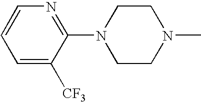 | 477 [M + H]⁺ |
| E35 | 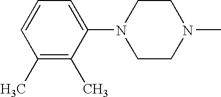 | 436 [M + H]⁺ |
| E36 | 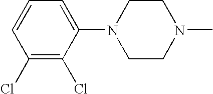 | 477/479/481 [M + H]⁺ |
| E37 | 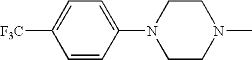 | 476 [M + H]⁺ |
| E38 | 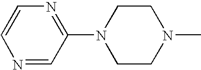 | 410 [M + H]⁺ |
| E39 | 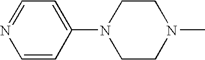 | 409 [M + H]⁺ |
| E40 | 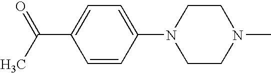 | 450 [M + H]⁺ |
| E41 | 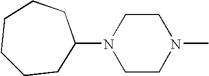 | 428 [M + H]⁺ |
| E42 | 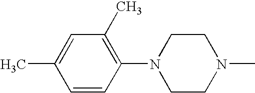 | 436 [M + H]⁺ |
| E43 | 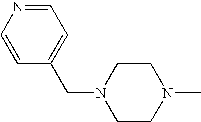 | 423 [M + H]⁺ |
| E44 | 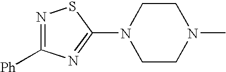 | 492 [M + H]⁺ |
| E45 | 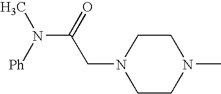 | 479 [M + H]⁺ |
| E46 | 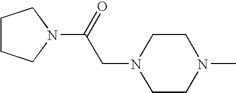 | 443 [M + H]⁺ |
| E47 | 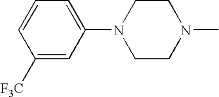 | 476 [M + H]⁺ |
| E48 | 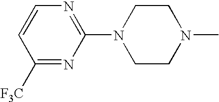 | 478 [M + H]⁺ |
| E49 | 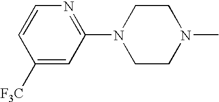 | 477 [M + H]⁺ |
| E50 | 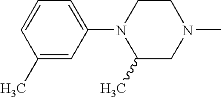 | 436 [M + H]⁺ |
| E51 | 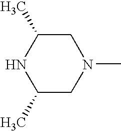 | 360 [M + H]⁺ |

### EXAMPLES 52-54

### E52-E54

Examples 52-54 (E52-E54) were prepared from 4-(3-piperidin-1-yl-propoxy)-2-trifluoromethyl-benzoyl chloride (D16) and the appropriate aryl piperazine according to the method described in Example 1 except that diethylaminomethyl polystyrene was employed as the base. The final products were purified by chromatography, and converted to the corresponding HCl salts with 1 M HCl in diethyl ether. All compounds displayed ¹H NMR and mass spectral data that were consistent with structure.

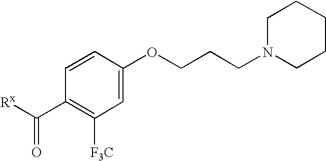

| Example | | Mass |
|---|---|---|
| No | R^{x} | Spectrum |
| E52 | 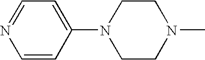 | 477 [M + H]⁺ |
| E53 | 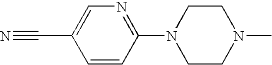 | 502 [M + H]⁺ |
| E54 | 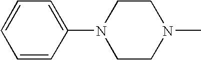 | 476 [M + H]⁺ |

### EXAMPLE 55

### N-[2,5-Difluoro-4-(3-piperidin-1-ylpropoxy)benzoyl]-4-phenylpiperazine dihydrochloride (E55)

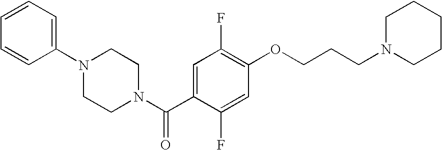

The title compound was prepared from 2,5-difluoro-4-(3-piperidin-1-ylpropoxy)benzoyl chloride hydrochloride (D19) and 4-phenylpiperazine according to the method described in Example 1 except that diethylaminomethyl polystyrene was employed as the base.

MS electrospray (+ ion) 444 (MH⁺).

### EXAMPLE 56

### N-[2-Fluoro-4-(3-Piperidin-1-ylpropoxy)benzoyl]-4-phenylpiperazine dihydrochloride (E56)

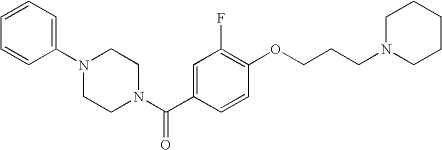

The title compound was prepared from 2-fluoro-4-(3-piperidin-1-ylpropoxy)benzoyl chloride hydrochloride (D22) and 4-phenylpiperazine according to the method described in Example 1 except that diethylaminomethyl polystyrene was employed as the base.

MS electrospray (+ ion) 426 (MH⁺).

### EXAMPLE 57

### 1-[4-(3-Piperidin-1-ylpropoxy)benzoyl]-4-(1-cyclohexanecarbonyl)-piperazine hydrochloride (E57)

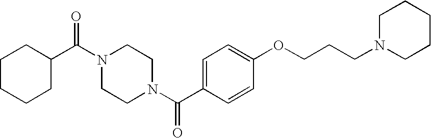

To 4-(3-piperidin-1-ylpropoxy)benzoyl chloride hydrochloride (D3) (0.24 g) in DCM (10 ml) was added 1-(cyclohexanecarbonyl)-piperazine (0.155 g) and diethylaminomethyl polystyrene (3.2 mmol/g, 0.699). The mixture was stirred for 16 h. The reaction mixture was then loaded directly onto a silica column and eluted with 0-10% MeOH (containing 10% 0.880 ammonia solution) in DCM. The isolated free base was dissolved in DCM (5 ml) and treated with 4N HCl/Dioxane solution (1 ml) with stirring for 10 min. The reaction was concentrated, and the residue co-evaporated with toluene (3×10 ml) and then dried at 50° C. under high vacuum for 16 h to yield the title compound (E57) as a pale solid (0.165 g). MS electrospray (+ ion)-442 (MH⁺).¹H NMR δ (DMSO-d₆): 9.71 (s, 1H), 7.39 (d, 2H, J=6.84 Hz), 7.00 (d, 2H, J=6.84 Hz), 4.10 (m, 2H), 3.47-3.25 (m, 10H), 3.16 (m, 2H), 2.90 (m, 2H), 2.55 (m, 1H), 2.19 (m, 2H), 1.82-1.62 (m, 10H), 1.40-1.16 (m, 6H).

### EXAMPLE 58

### 1-[4-(3-Piperidin-1-ylpropoxy)benzoyl]-4-(2-furoyl)-piperazine hydrochloride (E58)

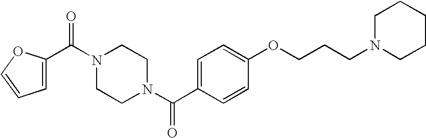

The title compound was prepared from 4-(3-piperidin-1-ylpropoxy)benzoyl chloride hydrochloride (D3) (0.24 g) and 1-(2-furoyl)piperazine (0.12 g) using the procedure described for Example 1 and isolated as a pale yellow solid (0.16 g). MS electrospray (+ ion) 426 (MH⁺).¹H NMR δ (DMSO-d₆): 9.80 (s, 1H), 7.84 (s, 1H), 7.43 (d, 2H, J=6.80 Hz), 7.03 (m, 1H), 7.02 (d, 2H, J=6.80 Hz), 6.63 (m, ₁H), 4.11 (m, 1H), 3.72-3.45 (m, 10H), 3.16 (m, 2H), 2.90 (m, 2H), 2.18 (m, 2H), 1.82-1.40 (m, 6H).

### EXAMPLE 59

### 1-[4-(3-Piperidin-1-ylpropoxy)benzoyl]-4-(thiophen-2-carbonyl)-piperazine Hydrochloride (E59)

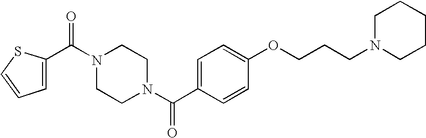

1-[4-(3-Piperidin-1-ylpropoxy)benzoyl]piperazine dihydrochloride (D5) (0.15 g) was stirred with diethylaminomethyl polystyrene (3.2 mmol/g, 0.35 g) in DCM (10 ml) and thiophen-2-carbonyl chloride (0.057 g) was added. The reaction was stirred for 16 h and then loaded directly onto a silica column, eluting with 0-10% MeOH (containing 10% 0.880 ammonia solution)/DCM. The isolated free base product was then dissolved in DCM (5 ml) and treated with 4N HCl/Dioxane solution (1 ml) and stirred for 10 min. The reaction was concentrated, and the residue co-evaporated with toluene (3×10 ml) then dried at 50° C. under high vacuum for 16 h to yield the title compound (E59) as a pale yellow solid (0.14 g). MS electrospray (+ ion) 442 (MH⁺). ¹H NMR δ (DMSO-6): 9.85 (s, 1H), 7.77 (m, 1H), 7.44 (m, 3H), 7.13 (m, 1H), 7.01 (d, 2H, 8.72 Hz), 4.10 (m, 2H), 3.70-3.34 (m, 10H), 3.17 (m, 1H), 2.89 (m, 2H), 2.17 (m, 2H), 1.79-1.37 (m, 6H).

### EXAMPLES 60-74

### E60-E74

Examples 60-74 were prepared from 1-[4-(3-piperidin-1-ylpropoxy)benzoyl]piperazine dihydrochloride (D5) and the appropriate acid chloride using the procedure described in Example 59 and displayed ¹H NMR and mass spectral data that were consistent with structure.

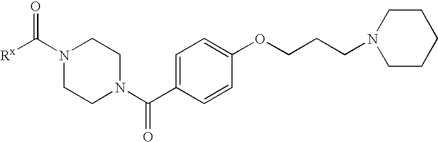

| Example | | Mass Spectrum |
|---|---|---|
| No | R^{x} | (ES⁺) |
| E60 | 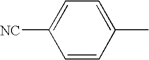 | [M + H]⁺ 461 |
| E61 | 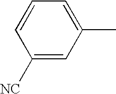 | [M + H]⁺ 461 |
| E62 | 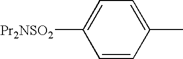 | [M + H]⁺ 600 |
| E63 | 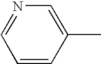 | [M + H]⁺ 437 |
| E64 | 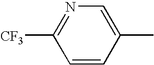 | [M + H]⁺ 505 |
| E65 | 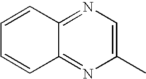 | [M + H]⁺ 488 |
| E66 | 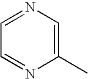 | [M + H]⁺ 452 |
| E67 | 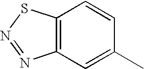 | [M + H]⁺ 494 |
| E68 | 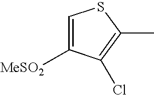 | [M + H]⁺ 555 |
| E69 | 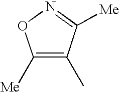 | [M + H]⁺ 455 |
| E70 | 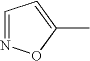 | [M + H]⁺ 427 |
| E71 | 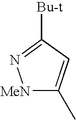 | [M + H]⁺ 496 |
| E72 | 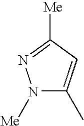 | [M + H]⁺ 454 |
| E73 | 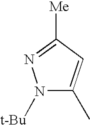 | [M + H]⁺ 496 |
| E74 | 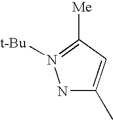 | [M + H]⁺ 496 |

### EXAMPLES 75-77

### E75-E77

Examples 75-77 were prepared from 1-[4-(3-piperidin-1-ylpropoxy)benzoyl]homopiperazine dihydrochloride (D7) and the appropriate carboxylic acid chloride or carbamoyl chloride following the procedure described for Example 59 and displayed ¹H NMR and mass spectral data that were consistent with structure.

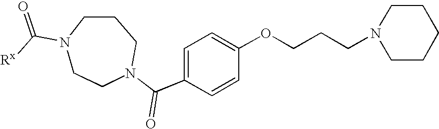

| Example | | Mass Spectrum |
|---|---|---|
| No | R^{x} | (ES⁺) |
| E75 | 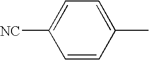 | [M + H]⁺ 475 |
| E76 | 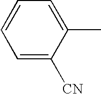 | [M + H]⁺ 475 |
| E77 | 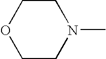 | [M + H]⁺ 459 |

### EXAMPLES 78 AND 79

### E78-E79

Examples 78 and 79 were prepared from (1S,4S)-2-[4-(3-piperidin-1-ylpropoxy)benzoyl]-2,5-diaza-bicyclo[2.2.1]heptane dihydrochloride (D9) and the appropriate acid chloride following the procedure described for Example 59 and displayed ¹H NMR and mass spectral data that were consistent with structure.

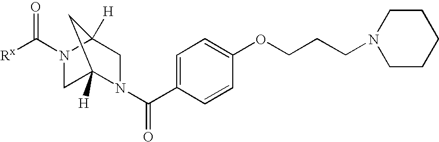

| Example | | Mass Spectrum |
|---|---|---|
| No | R^{x} | (ES⁺) |
| E78 | 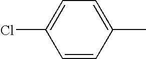 | [M + H]⁺ 483 |
| E79 | 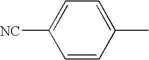 | [M + H]⁺ 473 |

### EXAMPLES 80 AND 81

### E80-E81

Examples 80 and 81 were prepared from (1S,4S)-2-[4-(3-piperidin-1-ylpropoxy)benzoyl]-2,5-diaza-bicyclo[2.2.1]heptane dihydrochloride (D9) and the appropriate carbamoyl chloride following the procedure described for Example 59, and displayed ¹H NMR and mass spectral data that were consistent with structure.

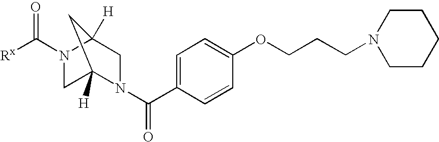

| Example | | Mass Spectrum |
|---|---|---|
| No | R^{x} | (ES⁺) |
| E80 | 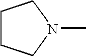 | [M + H]⁺ 441 |
| E81 | 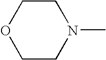 | [M + H]⁺ 457 |

### EXAMPLES 82-87

### E82-E87

Examples 82-87 were prepared from 1-[4-(3-piperidin-1-ylpropoxy)benzoyl]piperazine dihydrochloride (D5) and the appropriate carboxylic acid chloride using the procedure described in Example 59 and displayed ¹H NMR and mass spectral data that were consistent with structure.

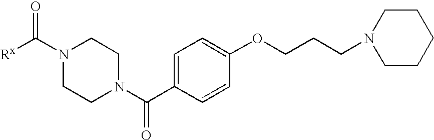

| Example | | Mass Spectrum |
|---|---|---|
| No | R^{x} | (ES⁺) |
| E82 | 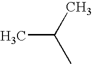 | [M + H]⁺ 402 |
| E83 | 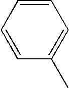 | [M + H]⁺ 436 |
| E84 | 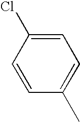 | [M + H]⁺ 471 |
| E85 | 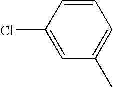 | [M + H]⁺ 471 |
| E86 | 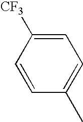 | [M + H]⁺ 504 |
| E87 | 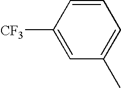 | [M + H]⁺ 504 |

### EXAMPLE 88

### 1-[4-(3-Piperidin-1-ylpropoxy)benzoyl]-4-(pyrrolidine-1-carbonyl)-piperazine Hydrochloride (E88)

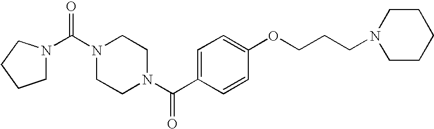

The title compound (E88) was prepared from 1-[4-(3-piperidin-1-ylpropoxy)benzoyl]piperazine dihydrochloride) (D5) (0.15 g) and pyrrolidine-1-carbonyl chloride (0.054 g) using the procedure described in Example 59 and was obtained as a white solid (0.10 g). MS electrospray (+ ion) 429 (MH⁺). ¹H NMR δ (DMSO-d₆): 9.75 (s, 1H), 7.40 (d, 2H, J=8.4 Hz), 7.00 (d, 2H, J=8.4 Hz), 4.10 (t, 2H, J=6.0 Hz), 3.47 (m, 6H), 3.27 (m, 4H), 3.18 (m, 6H), 2.87 (m, 2H), 2.17 (m, 2H), 1.74-1.39 (m, 10H).

### EXAMPLE 89

### 1-[4-(3-Piperidin-1-ylpropoxy)benzoyl]-4-(cycloheptanecarbonyl)-piperazine Hydrochloride (E89)

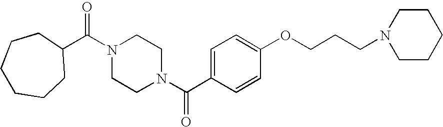

1-[4-(3-Piperidin-1-ylpropoxy)benzoyl]piperazine dihydrochloride (D5) (0.15 g) was dissolved in DCM (5 ml) and diethylaminomethyl polystyrene resin (3.2 mmol/g, 0.465 g) was added, followed by cycloheptane carboxylic acid (0.063 g), HOBT (0.065 g), and EDC (0.092 g). The reaction was stirred at rt overnight, then filtered and washed with saturated sodium hydrogen carbonate solution (3×50 ml) and brine (50 ml). The organic layer was dried (magnesium sulphate) and evaporated to give a crude product, which was purified by column chromatography [silica gel, eluted with 0-10% MeOH (containing 10% 0.880 ammonia solution) in DCM]. The isolated free base was then dissolved in DCM (5 ml) and treated with 4N HCl/dioxane solution (1 ml) and stirred for 10 min. The reaction was concentrated, and the residue co-evaporated with toluene (3×10 ml) then dried at 50° C. under high vacuum for 16 h to yield the title compound (E89) as a pale solid (0.051 g). MS electrospray (+ ion) 456 (MH⁺).¹H NMR δ (DMSO-d₆): 9.55 (s, 1H), 7.40 (d, 2H, J=8.76 Hz), 7.00 (d, 2H, J=8.76 Hz), 4.10 (t, 2H, J=9.93 Hz), 3.51 (m, 10H), 3.17 (m, 2H), 2.90 (m, 2H), 2.73 (m, 1H), 2.18 (m, 2H), 1.83-1.66 (m, 9H), 1.44 (m, 9H).

### EXAMPLES 90-99

### E90-E99

Examples 90-99 were prepared from 1-[4-(3-piperidin-1-ylpropoxy)benzoyl]piperazine dihydrochloride (D5) and the appropriate carboxylic acid using the procedure described in Example 89 and displayed ¹H NMR and mass spectral data that were consistent with structure.

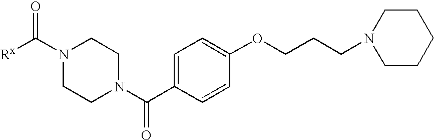

| Example | | Mass Spectrum |
|---|---|---|
| No | R^{x} | (ES⁺) |
| E90 | 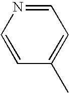 | [M + H]⁺ 437 |
| E91 | 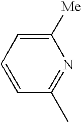 | [M + H]⁺ 451 |
| E92 | 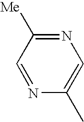 | [M + H]⁺ 452 |
| E93 | 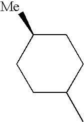 | [M + H]⁺ 456 |
| E94 | 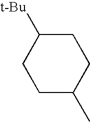 | [M + H]⁺ 498 |
| E95 | 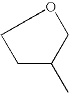 | [M + H]⁺ 430 |
| E96 | 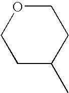 | [M + H]⁺ 444 |
| E97 | 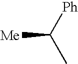 | [M + H]⁺ 464 |
| E98 | 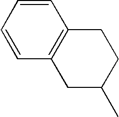 | [M + H]⁺ 490 |
| E99 | 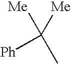 | [M + H]⁺ 478 |

### EXAMPLE 100

### (3R,5S)-1-[4-(3-Piperidin-1-ylpropoxy)benzoyl]-3,5-dimethyl-4-benzoyl-piperazine]hydrochloride (E100)

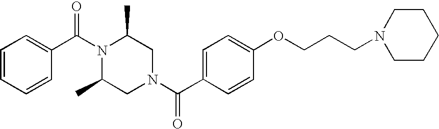

(3R,5S)-1-[4-(3-Piperidin-1-ylpropoxy)benzoyl]-3,5-dimethylpiperazine (D10) (0.15 g) was dissolved in DCM (5 ml) and treated with diethylaminomethyl polystyrene resin (3.2 mmol/g, 0.60 g) followed by benzoyl chloride (0.053 g). The reaction was stirred at rt for 16 h and then loaded directly onto a silica column, eluting with 0-10% MeOH (containing 10% 0.880 ammonia solution)/DCM. The isolated free base product was then dissolved in DCM (5 ml) and treated with 4N HCl/Dioxane solution (1 ml) and stirred for 10 min. The reaction was concentrated, and the residue co-evaporated with toluene (3×10 ml) then dried at 50° C. under high vacuum for 16 h to yield the title compound (E100) as a white solid (0.90 g). MS electrospray (+ ion) 464 (MH⁺).¹H NMR δ (DMSO-d6): 9.74 (1H, s), 7.39 (7H, m), 7.01 (2H, d, J=8.7 Hz), 4.40-4.09 (4H, m) 3.47-3.15 (6H, m), 2.92 (2H, m), 2.20-1.28 (10H, m), 1.15 (6H, m).

### EXAMPLES 101-102

### E101-E102

Examples 101-102 were prepared from (3R,5S)-1-[4-(3-piperidin-1-ylpropoxy)benzoyl]-3,5-dimethylpiperazine (D10) and the appropriate carboxylic acid chloride using the procedure described in Example 100 and displayed ¹H NMR and mass spectral data that were consistent with structure.

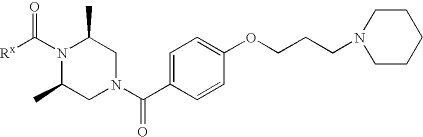

| Example | | Mass Spectrum |
|---|---|---|
| No | R^{X} | (ES⁺) |
| E101 | 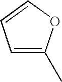 | [M + H]⁺ 454 |
| E102 | 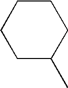 | [M + H]⁺ 470 |

### EXAMPLE 103

### (1S,4S)-5-[4-(3-Piperidin-1-ylpropoxy)benzoyl]-2,5-diaza-bicyclo[2.2.1]heptane-2 Carboxylic Acid t-Butyl Ester (E103)

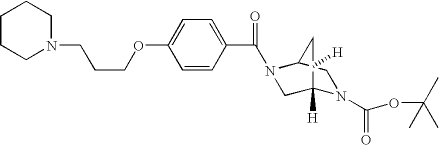

To (1S,4S)-2,5-diaza-bicyclo[2.2.1]heptane-2-carboxylic acid t-butyl ester (1.12 g) in DCM (10 ml) was added triethylamine (1.77 ml) and the reaction was cooled to 0° C. followed by the slow addition of 4-(3-piperidin-1-ylpropoxy)benzoyl chloride hydrochloride (D3) (1.8 g) in DCM (10 ml). The mixture was stirred at rt for 3 h, then washed with water. The organic layer was dried (MgSO₄) and evaporated to give the title compound (E103) as a cream coloured solid (2.52 g).

Mass Spectrum 444 [M+H]+

### EXAMPLE 104

### (1S,4S)-2-[4-(3-Piperidin-1-ylpropoxy)benzoyl]-2,5-diaza-bicyclo[2.2.1 heptane dihydrochloride (E104)

To (1S,4S)-5-[4-(3-piperidin-1-ylpropoxy)benzoyl]-2,5-diaza-bicyclo[2.2.1]heptane-2 carboxylic acid tert-butyl ester (E103) (2.52 g) in DCM (30 ml) was added 4N HCl (5 ml) and the mixture was allowed to stir at rt overnight. Evaporation of solvent followed by drying under high vacuum afforded the title compound (E104) as a foam (1.2 g).

### EXAMPLES 105-114

### E105-E114

Examples 105-114 were prepared from 1-[4-(3-piperidin-1-ylpropoxy)benzoyl]piperazine dihydrochloride (D5) and the appropriate acid using a similar procedure to that described in Example 89 and employing either DCM or DMF as solvent. All compounds displayed ¹H NMR and mass spectral data that were consistent with structure.

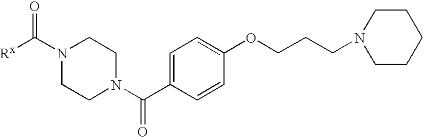

| Example | | Mass Spectrum |
|---|---|---|
| No | R^{X} | (ES⁺) |
| E105 | 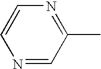 | [M + H]⁺ 338 |
| E106 | 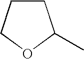 | [M + H]⁺ 430 |
| E107 | 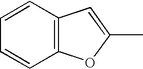 | [M + H]⁺ 476 |
| E108 | 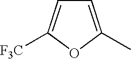 | [M + H]⁺ 494 |
| E109 | 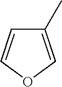 | [M + H]⁺ 426 |
| E110 | 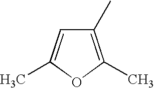 | [M + H]⁺ 454 |
| E111 | 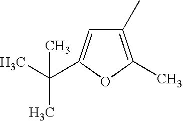 | [M + H]⁺ 496 |
| E112 | 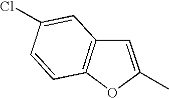 | [M + H]⁺ 511/513 |
| E113 | 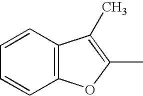 | [M + H]⁺ 490 |
| E114 | 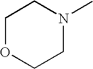 | [M + H]⁺ 445 |

### EXAMPLES 115-122

### E115-E122

Examples 115-122 were prepared using either Method A or B according to the table, and displayed ¹H NMR and mass spectral data that were consistent with structure.

Method A

1-[4-(3-Piperidin-1-ylpropoxy)-2-trifluoromethyl-benzoyl]piperazine dihydrochloride (D25) was reacted with the appropriate acid chloride following the method of Example 100 (E100). The isolated free base was converted into the hydrochloride salt and crystallised from acetone.

Method B

1-[4-(3-Piperidin-1-ylpropoxy)-2-trifluoromethyl-benzoyl]piperazine dihydrochloride (D25) was reacted with the appropriate carboxylic acid following the method of Example 89 (E89) except that DMF was employed as solvent. The isolated free base was converted into the hydrochloride salt and crystallised from acetone.

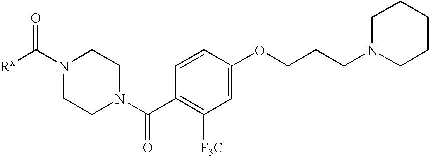

| Example | | Mass Spectrum | Synthetic |
|---|---|---|---|
| No | R^{X} | (ES⁺) | Method |
| E115 | 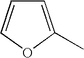 | [M + H]⁺ 494 | A |
| E116 | 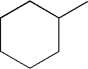 | [M + H]⁺ 510 | A |
| E117 | 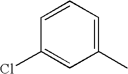 | [M + H]⁺ 539 | A |
| E118 | 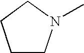 | [M + H]⁺ 497 | A |
| E119 | 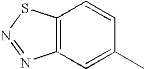 | [M + H]⁺ 562 | B |
| E120 | 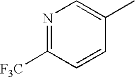 | [M + H]⁺ 573 | B |
| E121 | 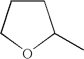 | [M + H]⁺ 498 | B |
| E122 | 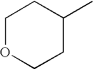 | [M + H]⁺ 512 | B |

### EXAMPLES 123 AND 124

### E123-E124

Examples 123 and 124 were prepared from (2R,6S)-2,6-dimethyl-1-[4-(3-piperidin-1-yl)propoxybenzoyl]piperazine dihydrochloride (D28) and the appropriate acid chloride using the method of Example 59 and displayed ¹H NMR and mass spectral data that were consistent with structure.

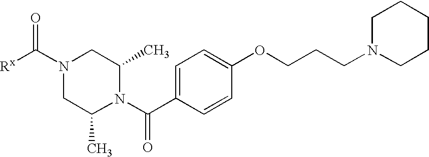

| Example | | Mass Spectrum |
|---|---|---|
| No | R^{X} | (ES⁺) |
| E123 | 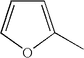 | [M + H]⁺ 454 |
| E124 | 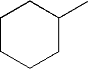 | [M + H]⁺ 470 |

### EXAMPLES 125-127

### E125-E127

Examples 125-127 were prepared from 4-[(1-isopropyl-4-piperidinyl)oxy]benzoyl]piperazine dihydrochloride (D38) and the appropriate acid chloride using the method of Example 59 and displayed ¹H NMR and mass spectral data that were consistent with structure.

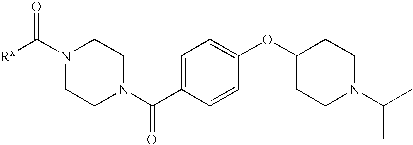

| Example | | Mass Spectrum |
|---|---|---|
| No | R^{X} | (ES⁺) |
| E125 | 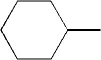 | [M + H]⁺ 442 |
| E126 | 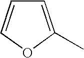 | [M + H]⁺ 426 |
| E127 | 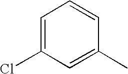 | [M + H]⁺ 471/473 |

### EXAMPLES 128-131

### E128-E131

Examples 128-131 were prepared from 4-[(1-isopropyl-4-piperidinyl)oxy]benzoyl]piperazine dihydrochloride (D38) and the appropriate acid using the method of Example 89 and displayed ¹H NMR and mass spectral data that were consistent with structure.

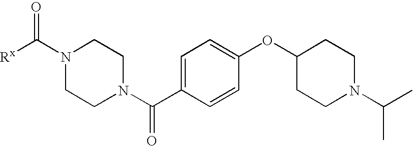

| Example | | Mass Spectrum |
|---|---|---|
| No | R^{X} | (ES⁺) |
| E128 | 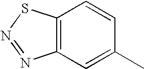 | [M + H]⁺ 494 |
| E129 | 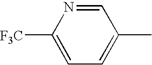 | [M + H]⁺ 505 |
| E130 | 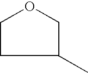 | [M + H]⁺ 430 |
| E131 | 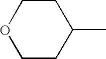 | [M + H]⁺ 444 |

### EXAMPLES 132-134

### E132-E134

Examples 132-134 were prepared from 4-[(1-cyclobutyl-4-piperidinyl)oxy]benzoyl]piperazine dihydrochloride (D37) and the appropriate acid chloride using the method of Example 59 and displayed ¹H NMR and mass spectral data that were consistent with structure.

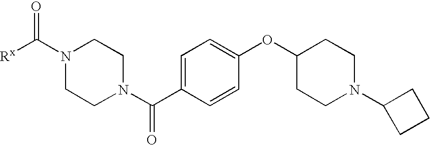

| Example | | Mass Spectrum |
|---|---|---|
| No | R^{X} | (ES⁺) |
| E132 | 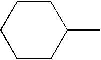 | [M + H]⁺ 454 |
| E133 | 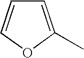 | [M + H]⁺ 438 |
| E134 | 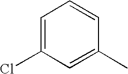 | [M + H]⁺ 483/485 |

### EXAMPLES 135-138

### E135-E138

Examples 135-138 were prepared from from 4-[(1-cyclobutyl-4-piperidinyl)oxy]benzoyl]piperazine dihydrochloride (D37) and the appropriate acid using the method of Example 89 except that DMF was used as solvent and displayed ¹H NMR and mass spectral data that were consistent with structure.

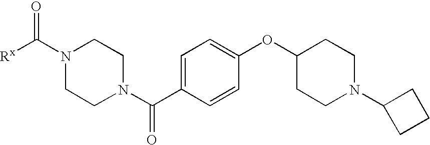

| Example | | Mass Spectrum |
|---|---|---|
| No | R^{X} | (ES⁺) |
| E135 | 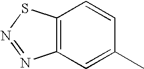 | [M + H]⁺ 506 |
| E136 | 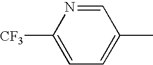 | [M + H]⁺ 517 |
| E137 | 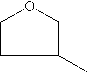 | [M + H]⁺ 442 |
| E138 | 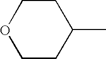 | [M + H]⁺ 456 |

### EXAMPLES 139-142

### E139-E142

Examples 139-142 were prepared from 4-[(1-cyclopentyl-4-piperidinyl)oxy]benzoyl]piperazine dihydrochloride (D39) and the appropriate acid chloride using the method of Example 59 and displayed ¹H NMR and mass spectral data that were consistent with structure.

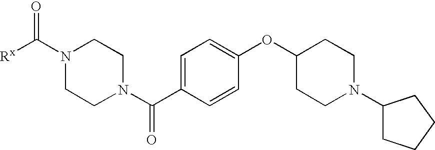

| Example | | Mass Spectrum |
|---|---|---|
| No | R^{X} | (ES⁺) |
| E139 | 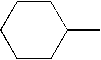 | [M + H]⁺ 468 |
| E140 | 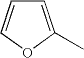 | [M + H]⁺ 452 |
| E141 | 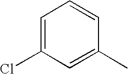 | [M + H]⁺ 497/499 |
| E142 | 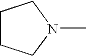 | [M + H]⁺ 455 |

### EXAMPLES 143-146

### E143-146

Examples 143-146 were prepared from from 4-[(1-cyclopentyl-4-piperidinyl)oxy]benzoyl]piperazine dihydrochloride (D39) and the appropriate acid using the method of of Example 89 except that DMF was used as solvent and displayed ¹H NMR and mass spectral data that were consistent with structure.

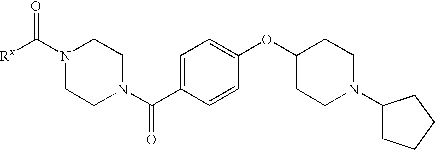

| Example | | Mass Spectrum |
|---|---|---|
| No | R^{X} | (ES⁺) |
| E143 | 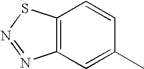 | [M + H]⁺ 520 |
| E144 | 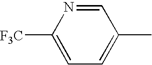 | [M + H]⁺ 531 |
| E145 | 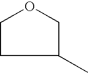 | [M + H]⁺ 456 |
| E146 | 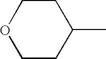 | [M + H]⁺ 470 |

### EXAMPLE 147

### N-[4-(3-Piperldin-1-ylpropoxy)benzoyl]-4-phenylpiperidine Hydrochloride (E147)

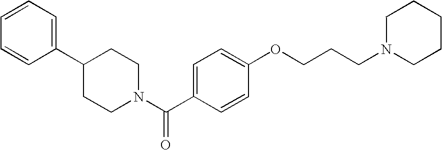

A solution of 4-(3-piperidin-1-ylpropoxy)benzoic acid hydrochloride (D2)(150 mg) in thionyl chloride (2 ml) was refluxed for 1 h, cooled to rt and evaporated. The acid chloride was re-evaporated from DCM (2×3 ml). The residue was redissolved in DCM (5 ml) and triethylamine (0.21 ml) and added to a stirred solution of 4-phenylpiperidine (81 mg) in DCM (2 ml) at rt. The mixture was stirred for 1 h and then chromatographed (silica gel, step gradient 4-8% MeOH in DCM). Fractions containing the required product were treated with excess hydrogen chloride (4M solution in dioxan) and then concentrated to yield the title compound (E147) (173 mg). MS electrospray (+ ion) 407 (MH⁺). ¹H NMR a (DMSO-d₆): 10.29 (1H, s), 7.41 (2H, d, J=8.5 Hz), 7.28 (5H, m), 6.99 (2H, d, J=8.5 Hz), 4.10 (2H, t, J=6.5 Hz), 2.70-3.53 (11H, m), 2.24 (2H, m), 1.30-1.85 (10H, m).

### EXAMPLE 148

### N-[4-(3-Piperidin-1-ylpropoxy)benzoyl]-4-(4-phenyl-1,3-dihydroimidazol-2-one-1-yl)piperidine Hydrochloride (E148)

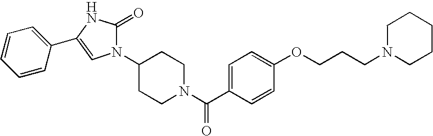

4-(3-Piperidin-1-ylpropoxy)benzoic acid hydrochloride (D2) (49 mg) was converted to the title compound (E148) by reaction with 4-phenyl-1,3-dihydroimidazol-2-one-1-ylpiperidine (Carling et al., J. Med. Chem., 1999, 42, 2706) (40 mg) using the method described in Example 1 (E1) (yield=73 mg). MS electrospray (+ ion) 490 (MH⁺). ¹H NMR δ (DMSO-d6): 10.73 (1H, s), 9.58 (1H, s), 6.96-7.55 (10H, m), 4.14 (2H, t, J=6 Hz), 3.25-3.77 (9H, m), 2.90 (2H, m), 2.17 (2H, m), 1.13-1.89 (10H, m).

### EXAMPLE 149

### N-[4-(3-Piperidin-1-ylpropoxy)benzoyl]piperidine Hydrochloride (E149)

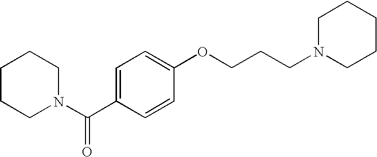

A solution of 4-(3-piperidin-1-ylpropoxy)benzoic acid hydrochloride (D2) (227 mg) in DMF at rt was treated with Argonaut PS Carbodiimide resin (778 mg, 1.3 mmol/g) and stirred for 5 min. Piperidine (0.05 ml) was added and the mixture stirred overnight, filtered and evaporated. The residue was partitioned between EtOAc (10 ml) and saturated sodium hydrogen carbonate solution (5 ml). The organic phase was collected, washed with water (3×), saturated brine, dried (MgSO₄) treated with excess hydrogen chloride (4M in dioxan) and evaporated to yield the title compound (E149) (72 mg). MS electrospray (+ ion) 331 (MH⁺). ¹H NMR δ (DMSO-d₆): 10.30 (1H, s), 7.33 (2H,d,J=8.8 Hz), 6.97 (2H,d,J=8.8 Hz), 4.10 (2H, t, J=6 Hz), 2.75-3.70 (10H, m), 2.20 (2H, m), 1.25-1.91 (12H, m).

### EXAMPLES 150-151

### E150-151

Examples 150-151 were prepared from 4-(3-piperidin-1-ylpropoxy)benzoic acid hydrochloride (D2) and the appropriate amine using the method outlined in Example 147 (E1) and displayed ¹H NMR and mass spectral data that were consistent with structure.

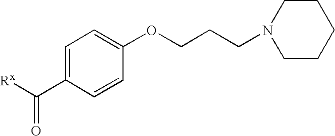

| Example | | Mass Spectrum |
|---|---|---|
| No | R^{X} | (ES⁺) |
| E150 | 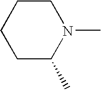 | 345 [M + H]⁺ |
| E151 | 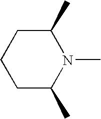 | 359 [M + H]⁺ |

### EXAMPLE 152

### E152

Example 152 was prepared from 4-(3-piperidin-1-ylpropoxy)benzoic acid hydrochloride (D2) and 4-hydroxy-4-phenylpiperidine using the method outlined in Example 147 (E147) with the exception that polymer supported base was employed. ¹H NMR and mass spectral data were consistent with structure.

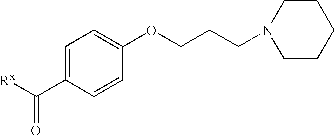

| Example | | |
|---|---|---|
| No | R^{X} | Mass Spectrum |
| E152 | 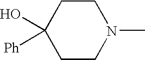 | 423 [M + H]⁺ |

### EXAMPLE 153

### N-[2-Fluoro-4-(3-piperidin-1-ylpropoxy)benzoyl]piperidine hydrochloride (E153)

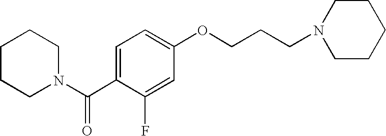

The title compound (E153) was prepared from 2-fluoro-4-(3-piperidin-1-ylpropoxy)benzoyl chloride hydrochloride (D22) and piperidine using the method described in Example 59. MS electrospray (+ ion) 349 (MH⁺)

### EXAMPLE 154

### N-[2,5-Difluoro-4-(3-piperidin-1-ylpropoxy)benzoyl]piperidine Hydrochloride (E154)

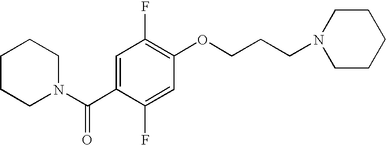

The tile compound (E154) was prepared from 2,5-difluoro-4-(3-piperidin-1-ylpropoxy)benzoyl chloride hydrochloride (D19) and piperidine using the method described in Example 59. MS electrospray (+ ion) 367 (MH⁺)

### EXAMPLE 155

### N-[2-Trifluoromethyl-4-(3-Piperidin-1-ylpropoxy)benzoyl]piperidine Hydrochloride (E155)

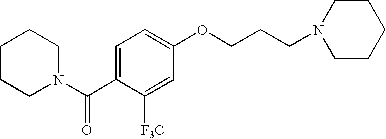

The tile compound (E155) was prepared from 4-(3-piperidin-1-yl-propoxy)-2-trifluoromethyl-benzoyl chloride hydrochloride (D16) and piperidine using the method described in Example 59. MS electrospray (+ ion) 399 (MH⁺)

### EXAMPLE 156

### (S)-N-[4-(3-Piperidin-1-ylpropoxy)benzoyl]-3-benzamidopyrrolidine Dihydrochloride (E156)

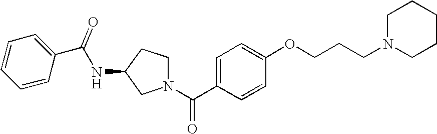

A stirred solution of (S)-N-[4-(3-piperidin-1-ylpropoxy)benzoyl]-3-aminopyrrolidine dihydrochloride (D11) (134 mg) and triethylamine (0.18 ml) in DCM at rt was treated with benzoyl chloride (0.046 ml). After 2 h the mixture was washed with saturated sodium hydrogen carbonate solution (5 ml), water (3×5 ml), dried (MgSO₄) and evaporated. The residue was chromatographed (silica gel, step gradient 0-20% MeOH in DCM). Fractions containing the required product were treated with excess hydrogen chloride (4M solution in dioxan) and then concentrated to yield the title compound (E156) (56 mg). MS electrospray (+ ion) 436 (MH⁺).¹H NMR δ (DMSO-d₆) at 353° K.: 10.15 (1H,s), 8.30 (1H,d,J=5.5 Hz), 7.82 (2H,d,J=8 Hz), 7.45 (5H,m), 6.97 (2H,d,J=8 Hz), 4.45 (1H,m), 4.12 (2H,t,J=6 Hz), 3.68 (2H,s), 2.80-3.90 (11H, m), 2.90 (2H,m), 2.18 (2H,m), 1.38-2.35 (6H,m).

### EXAMPLE 157

### N-[4-(3-Piperidin-1-ylpropoxy)benzoyl]-(R,S)-2-phenylpyrrolidine Hydrochloride (E157)

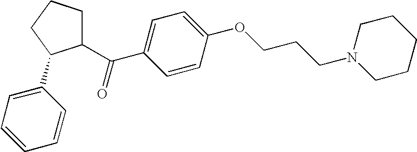

A solution of 4-(3-piperidin-1-ylpropoxy)benzoic acid hydrochloride (D2) (299 mg) in thionyl chloride (8 ml) was refluxed for 1 h, cooled to rt and evaporated. The acid chloride was re-evaporated from DCM (2×5 ml). The residue was redissolved in DCM (15 ml) and triethylamine (0.43 ml) and added to a stirred solution of (R,S)-2-phenylpyrrolidine (147 mg) in DCM (5 ml) at rt. The mixture was stirred for 1 h, washed with saturated sodium hydrogen carbonate solution (10 ml), water (3×10 ml), dried (MgSO₄) and evaporated. The residue was chromatographed (silica gel, step gradient 2-7% MeOH (containing 10% 0.880 ammonia solution) in DCM). Fractions containing the required product were treated with excess hydrogen chloride (4M solution in dioxan) and then concentrated to yield the title compound (E157) (332 mg). MS electrospray (+ ion) 393 (MH⁺). ¹H NMR δ (DMSO-d6): at 353° K. 10.20 (1H, s), 7.40 (2H, d, J=8.5 Hz), 7.25 (5H, m), 6.89(2H, d, J=8.5 Hz), 5.11 (1H, m), 4.09 (2H, t, J=6.5 Hz), 2.80-3.83 (6H, m), 2.05-2.55 (6H, m), 1.31-1.93 (8H, m).

### EXAMPLE 158

### (S)-N-[4-(3-Piperidin-1-ylpropoxy)benzoyl]-3-(naphthalene-1-carboxamidopyrrolidine dihydrochloride (E158)

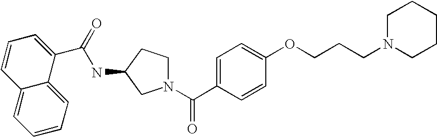

The title compound (E158) was prepared from (S)-N-[4-(3-piperidin-1-ylpropoxy)benzoyl]-3-aminopyrrolidine dihydrochloride (D11) and 1-naphthoyl chloride using the method outlined in Example 156. MS electrospray (+ ion) 486 (MH⁺). ¹H NMR data consistent with structure.

### EXAMPLE 159

### 4-Phenyl-9-[4-(3-piperidin-1-ylpropoxy)benzoyl]-1-oxa-4,9-diazaspiro-[5,5]-undecan-3-one hydrochloride (E159)

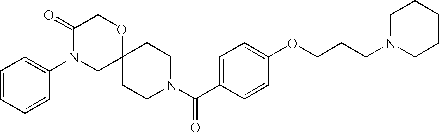

A solution of 4-(3-piperidin-1-ylpropoxy)benzoic acid hydrochloride (D2) (97 mg) in thionyl chloride (2.6 ml) was refluxed for 1 h, cooled to rt and evaporated. The acid chloride was re-evaporated from DCM (2×3 ml). The residue was redissolved in DCM (5 ml) and triethylamine (0.14 ml) and added to a stirred solution of 4-phenyl-1-oxa-4,9-diazaspiro-[5,5]-undecan-3-one (80 mg) (Caroon et al., J. Med. Chem., 1981, 24, 1320) in DCM (2 ml) at rt. The mixture was stirred for 1 h, washed with saturated sodium hydrogen carbonate solution (5 ml), water (3×5 ml), dried (MgSO₄) and evaporated. The residue was chromatographed [silica gel, step gradient 0-5% MeOH (containing 10% of 0.880 ammonia solution) in DCM]. Fractions containing the required product were treated with excess hydrogen chloride (4M solution in dioxan) and then concentrated to yield the title compound (E159) (79 mg). MS electrospray (+ ion) 492 (MH⁺). ¹H NMR δ (DMSO-d6): 9.77 (1H, s), 6.98-7.44 (9H, m), 4.25 (2H, s), 4.10 (2H, t, J=6 Hz), 3.68 (2H, s), 3.05-3.78 (8H, m), 2.90 (2H, m), 2.18 (2H, m), 1.28-2.05 (10H, m).

### EXAMPLE 160

### 3-Benzyl-8-[4-(3-piperidin-1-ylpropoxy)benzoyl]-1,3,8-triaza-spiro[4.5]-decan-2-one (E160)

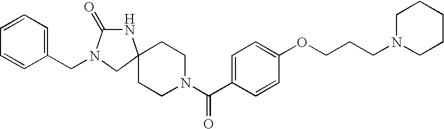

4-(3-Piperidin-1-ylpropoxy)benzoic acid hydrochloride (D2) (49 mg) was converted to the title compound (E160) by reaction with 3-benzyl-1,3,8-triaza-spiro[4.5]decan-2-one (Smith et al., J. Med. Chem., 1995, 38, 3772) (40 mg) using the method described in Example 159 (E159) with the exception that the product was isolated as the free base. (yield=47 mg). MS electrospray (+ ion) 491 (MH⁺). ¹H NMR δ (CDCl₃): 6.86-7.42 (9H, m), 4.88 (1H, s), 4.39 (2H, s), 4.00 (2H, t, J=6.4 Hz), 3.65 (4H, m), 3.14 (2H, s), 2.45 (2H, m), 1.98 (2H, m), 1.37-1.82 (10H, m).

### EXAMPLES 161-162

### E161-162

Examples 161-162 were prepared from 4-(3-piperidin-1-ylpropoxy)benzoic acid hydrochloride (D2) and the appropriate amine using the method outlined in Example 159 (E159) and displayed ¹H NMR and mass spectral data that were consistent with structure.

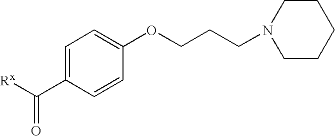

| Example | | Mass Spectrum |
|---|---|---|
| No | R^{X} | (ES⁺) |
| E161 | 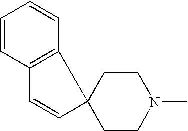 | 431 [M + H]⁺ |
| E162 | 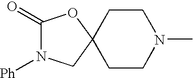 | 478 [M + H]⁺ |

### EXAMPLE 163

### N-[4-(3-Piperidin-1-ylpropoxy)benzenesulfonyl]morpholine Hydrochloride (E163)

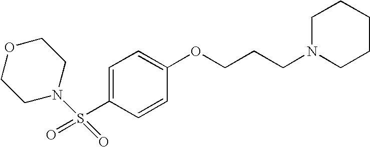

A solution of 4-[4-(3-bromopropoxy)benzenesulfonyl]morpholine (D13) (96 mg) in 1-butanol (5 ml) and piperidine (0.22 ml) was heated at 100° C. for 16 h, cooled to rt and evaporated. The residue was redissolved in EtOAc (10 ml), washed with saturated sodium hydrogen carbonate solution (5 ml), water (3×5 ml), dried (MgSO₄) and evaporated. The residue was redissolved in DCM and treated with excess hydrogen chloride (4M solution in dioxan) and then concentrated to yield the title compound (E163) (75 mg). MS electrospray (+ ion) 369 (MH⁺). ¹H NMR δ (DMSO-d₆): 10.21 (1H, s), 7.68 (2H, d, J=8.8 Hz), 7.18 (2H, d, J=8.8 Hz), 4.18 (2H, t, J=6 Hz), 3.62 (2H, m), 3.44 (2H, m), 3.17 (2H, m), 2.84 (6H, m), 1.30-1.85 (6H, m).

### EXAMPLES 164-168

### E164-168

Examples 164-168 were prepared from the appropriate amine using an analogous method to that described in Description 13 (D13) followed by Example 163 (E163). All compounds displayed ¹H NMR and mass spectral data that were consistent with structure.

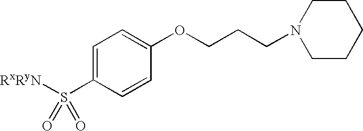

| Example | | |
|---|---|---|
| No | R^{X}R^{Y}N | Mass Spectrum |
| E164 | 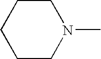 | 367 [M + H]⁺ |
| E165 | 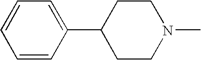 | 443 [M + H]⁺ |
| E166 | 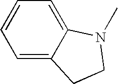 | 401 [M + H]⁺ |
| E167 | 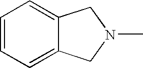 | 401 [M + H]⁺ |
| E168 | 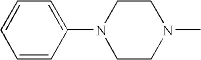 | 444 [M + H]⁺ |

### EXAMPLE 169

### N-[4-(3-Piperidin-1-ylpropoxy)benzenesulfonyl]piperazine dihydrochloride (E169)

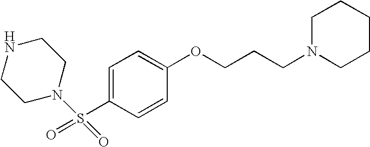

The title compound (E169) was prepared using an analogous method to that described in Description 13 (D13) followed by Example 163 (E163) by treating N-Boc piperazine with 1-bromo-3-(4-chlorosulfonylphenoxy)propane followed by reaction with piperidine. Subsequent deprotection with HCl afforded the dihydrochloride salt. MS electrospray (+ ion) 368 (MH⁺).

### EXAMPLES 170-171

### E170-171

Examples 170-171 were prepared from Example 169 (E169) by treatment with the appropriate acid chloride in the presence of triethylamine using DCM as solvent.

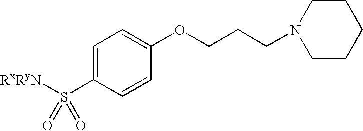

| Example | | |
|---|---|---|
| No | R^{X}R^{Y}N | Mass Spectrum |
| E170 | 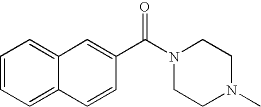 | 522 [M + H]⁺ |
| E171 | 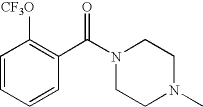 | 556 [M + H]⁺ |

### EXAMPLE 172

### 1-[4-(3-Piperldin-1-ylpropoxy)benzoyl]-4-t-butoxycarbonylhomopiperazine (E172)

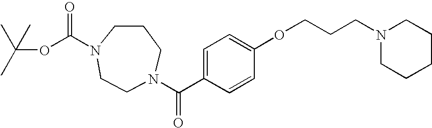

To t-butoxycarbonylhomopiperazine (0.76 g) in DCM (10 ml) was added triethylamine (1.2 ml) and the mixture was cooled to 0° C. followed by the slow addition of 4-(3-piperidin-1-ylpropoxy)benzoyl chloride hydrochloride (D3) (1.2 g) in DCM (10 ml). The mixture was stirred at rt for 3 h, then washed with water. The organic layer was dried (MgSO₄) and evaporated to give the title compound (E172) as a cream coloured solid (1.69 g).

Mass Spectrum 446 [M+H]⁺

Abbreviations

Boc tertbutoxycarbonyl
EtOAc ethyl acetate
h hour
DCM dichloromethane
MeOH methanol
rt room temperature
DCC dicyclohexylcarbodiimide
DMF dimethylformamide

All publications, including but not limited to patents and patent applications, cited in this specification are herein incorporated by reference as if each individual publication were specifically and individually indicated to be incorporated by reference herein as though fully set forth.

Biological Data

A membrane preparation containing histamine H3 receptors may be prepared in accordance with the following procedures:

(i) Generation of Histamine H3 Cell Line

DNA encoding the human histamine H3 gene (Huvar, A. et al. (1999) Mol. Pharmacol. 55(6), 1101-1107) was cloned into a holding vector, pcDNA3.1 TOPO (InVitrogen) and its cDNA was isolated from this vector by restriction digestion of plasmid DNA with the enzymes BamHI and Not-1 and ligated into the inducible expression vector pGene (InVitrogen) digested with the same enzymes. The GeneSwitch™ system (a system where in transgene expression is switched off in the absence of an inducer and switched on in the presence of an inducer) was performed as described in U.S. Pat. Nos. 5,364,791; 5,874,534; and 5,935,934. Ligated DNA was transformed into competent DH5α E. coli host bacterial cells and plated onto Luria Broth (LB) agar containing Zeocin™ (an antibiotic which allows the selection of cells expressing the sh ble gene which is present on pGene and pSwitch) at 50 μg ml⁻¹. Colonies containing the religated plasmid were identified by restriction analysis. DNA for transfection into mammalian cells was prepared from 250 ml cultures of the host bacterium containing the pGeneH3 plasmid and isolated using a DNA preparation kit (Qiagen Midi-Prep) as per manufacturers guidelines (Qiagen).

CHO K1 cells previously transfected with the pSwitch regulatory plasmid (InVitrogen) were seeded at 2×10e6 cells per T75 flask in Complete Medium, containing Hams F12 (GIBCOBRL, Life Technologies) medium supplemented with 10% v/v dialysed foetal bovine serum, L-glutamine, and hygromycin (100 μg ml⁻¹), 24 hours prior to use. Plasmid DNA was transfected into the cells using Lipofectamine plus according to the manufacturers guidelines (InVitrogen). 48 hours post transfection cells were placed into complete medium supplemented with 500 μg ml⁻¹ Zeocin™.

10-14 days post selection 10 nM Mifepristone (InVitrogen), was added to the culture medium to induce the expression of the receptor. 18 hours post induction cells were detached from the flask using ethylenediamine tetra-acetic acid (EDTA; 1:5000; InVitrogen), following several washes with phosphate buffered saline pH 7.4 and resuspended in Sorting Medium containing Minimum Essential Medium (MEM), without phenol red, and supplemented with Earles salts and 3% Foetal Clone II (Hyclone).

Approximately 1×10e7 cells were examined for receptor expression by staining with a rabbit polyclonal antibody, 4a, raised against the N-terminal domain of the histamine H3 receptor, incubated on ice for 60 minutes, followed by two washes in sorting medium.

Receptor bound antibody was detected by incubation of the cells for 60 minutes on ice with a goat anti rabbit antibody, conjugated with Alexa 488 fluorescence marker (Molecular Probes). Following two further washes with Sorting Medium, cells were filtered through a 50 μm Filcon™ (BD Biosciences) and then analysed on a FACS Vantage SE Flow Cytometer fitted with an Automatic Cell Deposition Unit. Control cells were non-induced cells treated in a similar manner. Positively stained cells were sorted as single cells into 96-well plates, containing Complete Medium containing 500 μg ml⁻¹ Zeocin™ and allowed to expand before reanalysis for receptor expression via antibody and ligand binding studies. One clone, 3H3, was selected for membrane preparation.

(ii) Membrane Preparation from Cultured Cells

All steps of the protocol are carried out at 4° C. and with pre-cooled reagents. The cell pellet is resuspended in 10 volumes of buffer A2 containing 50 mM N-2-hydroxyethylpiperazine-N′-2-ethanesulfonic acid (HEPES) (pH 7.40) supplemented with 10e4M leupeptin (acetyl-leucyl-leucyl-arginal; Sigma L2884), 25 μg/ml bacitracin (Sigma B0125), 1 mM ethylenediamine tetra-acetic acid (EDTA), 1 mM phenylmethylsulfonyl fluoride (PMSF) and 2×10e-6M pepstain A (Sigma). The cells are then homogenised by 2×15 second bursts in a 1 litre glass Waring blender, followed by centrifugation at 500 g for 20 minutes. The supernatant is then spun at 48,000 g for 30 minutes. The pellet is resuspended in 4 volumes of buffer A2 by vortexing for 5 seconds, followed by homogenisation in a Dounce homogeniser (10-15 strokes). At this point the preparation is aliquoted into polypropylene tubes and stored at −70° C.

Compounds of the invention may be tested for in vitro biological activity in accordance with the following assays:

(I) Histamine H3 Binding Assay

For each compound being assayed, in a white walled clear bottom 96 well plate, is added:
(a) 10 μl of test compound (or 10 μl of iodophenpropit (a known histamine H3 antagonist) at a final concentration of 10 mM) diluted to the required concentration in 10% DMSO;
(b) 10 μl ¹²⁵I 4-[3-(4-iodophenylmethoxy)propyl]-1H-imidazolium (iodoproxyfan) (Amersham; 1.85 MBq/μl or 50 μCi/ml; Specific Activity ˜2000Ci/mmol) diluted to 200 pM in assay buffer (50 mM Tris(hydroxymethyl)aminomethane buffer (TRIS) pH 7.4, 0.5 mM ethylenediamine tetra-acetic acid (EDTA)) to give 20 pM final concentration; and
(c) 80 μl bead/membrane mix prepared by suspending Scintillation Proximity Assay (SPA) bead type WGA-PVT at 100 mg/ml in assay buffer followed by mixing with membrane (prepared in accordance with the methodology described above) and diluting in assay buffer to give a final volume of 80 μl which contains 7.5 μg protein and 0.25 mg bead per well—mixture was pre-mixed at room temperature for 60 minutes on a roller. The plate is shaken for 5 minutes and then allowed to stand at room temperature for 3-4 hours prior to reading in a Wallac Microbeta counter on a 1 minute normalised tritium count protocol. Data was analysed using a 4-parameter logistic equation.
   (II) Histamine H3 Functional Antagonist Assay

For each compound being assayed, in a white walled clear bottom 96 well plate, is added:—
(a) 10 μl of test compound (or 10 μl of guanosine 5′-triphosphate (GTP) (Sigma) as non-specific binding control) diluted to required concentration in assay buffer (20 mM N-2-Hydroxyethylpiperazine-N′-2-ethanesulfonic acid (HEPES)+100 mM NaCl+10 mM MgCl₂, pH7.4 NaOH);
(b) 60 μl bead/membrane/GDP mix prepared by suspending wheat germ agglutinin-polyvinyltoluene (WGA-PVT) scintillation proximity assay (SPA) beads at 100 mg/ml in assay buffer followed by mixing with membrane (prepared in accordance with the methodology described above) and diluting in assay buffer to give a final volume of 60 μl which contains 10 μg protein and 0.5 mg bead per well—mixture is pre-mixed at 4° C. for 30 minutes on a roller and just prior to addition to the plate, 10 μM final concentration of guanosine 5′ diphosphate (GDP) (Sigma; diluted in assay buffer) is added; The plate is incubated at room temperature to equilibrate antagonist with receptor/beads by shaking for 30 minutes followed by addition of:
(c) 10 μl histamine (Tocris) at a final concentration of 0.3 μM; and
(d) 20 μl guanosine 5′[γ35-S] thiotriphosphate, triethylamine salt (Amersham; radioactivity concentration=37 kBq/μl or 1 mCi/ml; Specific Activity 1160 Ci/mmol) diluted to 1.9 nM in assay buffer to give 0.38 nM final.

The plate is then incubated on a shaker at room temperature for 30 minutes followed by centrifugation for 5 minutes at 1500 rpm. The plate is read between 3 and 6 hours after completion of centrifuge run in a Wallac Microbeta counter on a 1 minute normalised tritium count protocol. Data is analysed using a 4-parameter logistic equation. Basal activity used as minimum i.e. histamine not added to well.

Results

The compounds of Examples E1-E103 and E105-E172 were tested in the histamine H3 functional antagonist assay and exhibited pK_{b} values >7.5. More particularly, the compounds of Examples E1-3, E5-7, E9, Eli, E13-16, E18-19, E21-25, E28, E30, E33, E35, E3741, E47, E49, E51-53, E57, E59-61, E63-65, E67-68, E72, E75, E78, E80, E84-86, E88-89, E93-94, E96, E98, E99-E101, E107-108, E110-111, E115-119, E121-122, E123, E125, E128-131, E132-138, E139-146, E149-151, E155-160, E162, E164-165, E170 exhibited pK_{b} values >8.5.

What is claimed is:

1. A compound of formula (I) or a pharmaceutically acceptable salt thereof: 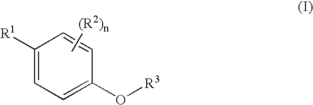
wherein:
R¹ represents a group of formula (A): 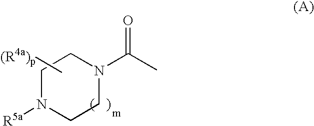
wherein R^{4a} represents C₁₋₆ alkyl, oxo, aryl, heteroaryl or heterocyclyl;
R^{5a} represents hydrogen, -C₁ alkyl, -C₁₋₆ alkylC₁₋₆ alkoxy, -C₁₋₆ alkoxycarbonyl, -C₃₋₈ cycloalkyl, -aryl, -heterocyclyl, heteroaryl, -C₁ alkyl-aryl, —CH(aryl)(aryl), -C₁₋₆ alkyl-C₃₋₈ cycloalkyl, -C₁₋₆ alkyl-heteroaryl or -C₁ alkyl-heterocyclyl,
wherein R^{5a} may be optionally substituted by one or more substituents which may be the same or different, and which are selected from the group consisting of halogen, hydroxy, cyano, nitro, oxo, haloC₁₋₆ alkyl, polyhaloC₁₋₆ alkyl, haloC₁₋₆ alkoxy, polyhaloC₁₋₆alkoxy, C₁₋₆ alkyl, C₁₋₆alkoxy, C₁₋₆ alkylthio, C₁₋₆ alkoxyC₁₋₆ alkyl, C₃₋₇ cycloalkylC₁₋₆ alkoxy, C₁₋₆alkanoyl, C₁₋₆ alkoxycarbonyl, C₁₋₆ alkylsulfonyl, C₁-alkylsulfinyl, C₁₋₆ alkylsulfonyloxy, C₁₋₆ alkylsulfonylC₁₋₆ alkyl, C₁₋₆alkylsulfonamidoC₁₋₆alkyl, C₁₋₆ alkylamidoC₁₋₆alkyl or a group NR^{15a}R^{16a}, —CONR^{15a}R^{16a}, —NR^{15a}COR^{16a}, —NR^{15a}SO₂R^{16a} or —SO₂NR^{15a}R^{16a}, wherein R^{15a} and R^{16a} independently represent hydrogen, C₁₋₆ alkyl, aryl or together with the nitrogen to which they are attached may form a nitrogen containing heterocyclyl group;
m is 1 or 2;
p is 0, 1, 2 or 3, or when p represents 2, said R^{4a} groups may instead form a bridging group consisting of one or two methylene groups;
or R¹ represents a group of formula (B): 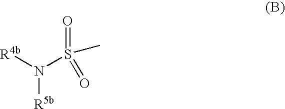
wherein NR^{4b}R^{5b} represents an N-linked -heterocyclyl, -heterocyclyl-X^{b}-aryl, -heterocyclyl-X^{b}-heteroaryl, -heterocyclyl-X^{b}-heterocyclyl, -heteroaryl, -heteroaryl-X^{b}-aryl, -heteroaryl-X^{b}-heteroaryl or -heteroaryl-X^{b}-heterocyclyl group;
wherein said aryl, heteroaryl and heterocyclyl groups of NR^{4b}R^{5b} may be optionally substituted by one or more substituents which may be the same or different, and which are selected from the group consisting of halogen, hydroxy, cyano, nitro, oxo, haloC₁₋₆ alkyl, polyhaloC₁₋₆ alkyl, haloC₁₋₆ alkoxy, polyhaloC₁₋₆ alkoxy, C₁₋₆ alkyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆alkoxy, C₁₋₆alkanoyl, C₁₋₆ alkoxycarbonyl, arylC₁₋₆ alkyl, heteroarylC₁₋₆ alkyl, C₁₋₆ alkylsulfonyl, C₁₋₆alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, aryloxy, C₁₋₆ alkylsulfonamidoC₁₋₆ alkyl, C₁₋₆ alkylamidoC₁₋₆ alkyl, arylsulfonamido, arylaminosulfonyl, arylsulfonamidoC₁₋₆ alkyl, arylcarboxamidoC₁₋₆ alkyl, aroylC₁₋₆ alkyl, arylC₁₋₆ alkanoyl, or a group —NR^{15b}R^{16b}, —CONR^{15b}R^{16b}, —NR^{5b}COR^{16b}, —NR^{15b}SO₂R^{16b} or —SO₂NR^{15b}R^{16b}, wherein R^{15b} and R^{16b} independently represent hydrogen or C₁₋₆alkyl;
X^{b} represents a bond, CO, NHCO or CONH;
or R¹ represents a group of formula (C): 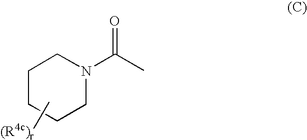
wherein R^{4c} represents C₁₋₆ alkyl, OH, aryl or heterocyclyl, wherein said aryl and heterocyclyl groups may be optionally substituted by halogen, C₁₋₆ alkyl, C₁₋₆ alkoxy, cyano, amino, oxo, trifluoromethyl or an aryl group;
r is 0, 1 or 2;
or R¹ represents a group of formula (D): 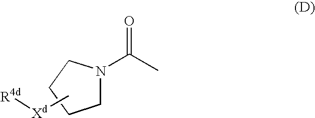
wherein R^{4d} represents aryl or heteroaryl wherein said aryl and heteroaryl groups may be optionally substituted by one or more substituents which may be the same or different, and which are selected from the group consisting of halogen, C₁₋₆ alkyl, C₁₋₆ alkoxy, cyano, amino or trifluoromethyl;
X^{d} represents a bond or NHCO, such that when X^{d} represents NHCO, the group R^{4d}—X^{d} is attached at the 3-position of the pyrrolidinyl ring;
or R¹ represents a group of formula —CO-E, wherein E represents a group of formula E^{a}, E^{b} or E^{c}: 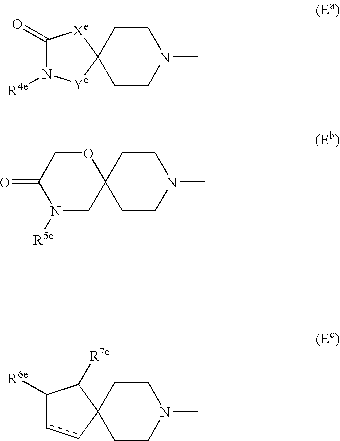
wherein X^{e} represents O or N—R^{8e};
Y^{e} represents —C(HR^{9e})— or —C(═O)—;
R^{4e}, R^{5e}, R^{8e} and R^{9e} independently represent hydrogen, C₁ alkyl, aryl, heteroaryl, —C₁₋₆alkyl-aryl or -C₁₋₆alkyl-heteroaryl;
R^{6e} and R^{7e} independently represent hydrogen, C₁₋₆ alkyl, aryl, heteroaryl, -C₁₋₆ alkyl-aryl, -C₁₋₆ alkyl-heteroaryl or R^{6e} and R^{7e} together with the carbon atoms to which they are attached may form a benzene ring; [symbol]
is a single or double bond;
wherein said aryl or heteroaryl groups of R^{4e}, R^{5e}, R^{6e}, R^{7e} and R^{9e} may be optionally substituted by one or more substituents which may be the same or different, and which are selected from the group consisting of C₁₋₆ alkyl, CF₃, C₁₋₆ alkoxy, halogen, cyano, sulfonamide or C₁₋₆ alkylsulfonyl;
or R¹ represents a group of formula (F): 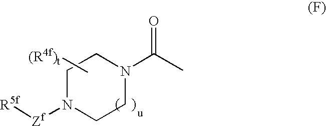
wherein t is 0, 1 or 2;
u is 1 or 2;
R^{4f} represents C₁₋₆ alkyl or when t represents 2, said R^{4f} groups may instead form a bridging group consisting of one or two methylene groups;
R^{5f} represents -C₁₋₆ alkyl, -C₁₋₆ alkylC₁₋₆alkoxy, -C₁₋₆ cycloalkyl, aryl, heterocyclyl, heteroaryl, -C₁₋₆ alkyl-aryl, -C₁₋₆ alkyl-C₃₋₈ cycloalkyl, -C₁₋₆ alkyl-heteroaryl, -C₁₋₆ alkyl-heterocyclyl, -aryl-aryl, -aryl-heteroaryl, -aryl-heterocyclyl, -heteroaryl-aryl, -heteroaryl-heteroaryl, -heteroaryl-heterocyclyl, -heterocyclyl-aryl, -heterocyclyl-heteroaryl or -heterocyclyl-heterocyclyl;
wherein R^{5f} may be optionally substituted by one or more substituents which may be the same or different, and which are selected from the group consisting of halogen, hydroxy, cyano, nitro, oxo, haloC₁₋₆alkyl, polyhaloC₁₋₆alkyl, haloC₁₋₆alkoxy, polyhaloC₁₋₆alkoxy, C₁— alkyl, C₁₋₆alkoxy, C₁₋₆alkylthio, C₁₋₆alkoxyC₁₋₆alkyl, C₃₋₇ cycloalkylC₁₋₆alkoxy, C₁₋₆alkanoyl, C₁₋₆alkoxycarbonyl, C₁₋₆alkylsulfonyl, C₁₋₆alkylsulfinyl, C₁₋₆alkylsulfonyloxy, C₁₋₆alkylsulfonylC₁₋₆alkyl, C₁₋₆alkylsulfonamidoC₁₋₆alkyl, C₁₋₆alkylamidoC₁₋₆alkyl, arylsulfonyl, arylsulfonyloxy, aryloxy, arylsulfonamido, arylcarboxamido, aroyl, or a group NR^{15f}R^{16f}, —CONR^{15f}R¹⁶f, —NR^{15f}COR^{16f}, —NR¹⁵SO₂R^{16f} or —SO₂NR^{15f}R^{16f}, wherein R^{15f} and R^{16f} independently represent hydrogen or C₁₋₆alkyl or together form a heterocyclic ring;
Z^{f} represents CO or SO₂;
R² represents halogen, C₁₋₆alkyl, C₁₋₆alkoxy, cyano, amino or trifluoromethyl;
n is 0, 1 or 2;
R³ represents —(CH₂)_{q}—NR¹¹R¹² or a group of formula (i): 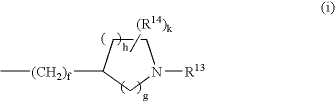
wherein q is 2, 3 or 4;
R¹¹ and R¹² independently represent C₁₋₆alkyl or together with the nitrogen atom to which they are attached represent an N-linked heterocyclic group selected from pyrrolidine, piperidine and homopiperidine optionally substituted by one or two R¹⁷ groups;
R¹³ represents C₁₋₆alkyl, C₃₋₆ cycloalkyl or -C₁₋₆ alkyl-C₃₋₆cycloalkyl;
R¹⁴ and R¹⁷ independently represent halogen, C₁₋₆alkyl, haloC₁₋₆ alkyl, OH, diC₁₋₆ alkylamino or C₁— alkoxy;
f and k independently represent 0, 1 or 2;
g is 0, 1 or 2 and h is 0, 1, 2 or 3, such that g and h cannot both be 0;
or solvates thereof.

2. A compound according to claim 1 which is a compound selected from the group consisting of E1-E172 or a pharmaceutically acceptable salt thereof.

3. A pharmaceutical composition which comprises the compound of formula (I) as defined in claim 1 or a pharmaceutically acceptable salt thereof and a pharmaceutically acceptable carrier or excipient.

4-6. (canceled)

7. A method of treatment of neurological diseases which comprises administering to a host in need thereof an effective amount of a compound of formula (I) as defined in claim 1 or a pharmaceutically acceptable salt thereof.

8. (canceled)
